(19) United States
(12) Patent Application Publication
(10) Pub. No.: US 2016/0309686 A1
(43) Pub. Date: Oct. 27, 2016

(54) TRANSGENIC BIRDS THAT PRODUCE CHIMERIC HUMAN IMMUNOGLOBULINS

(71) Applicants: Philip A. Leighton, San Francisco, CA (US); Emily J. Cadera, San Francisco, CA (US)

(72) Inventors: Philip A. Leighton, San Francisco, CA (US); Emily J. Cadera, San Francisco, CA (US)

(73) Assignee: Synageva BioPharma Corp., Lexington, MA (US)

(21) Appl. No.: 15/142,097

(22) Filed: Apr. 29, 2016

Related U.S. Application Data
(63) Continuation of application No. 14/240,099, filed on Jun. 23, 2014, now abandoned.
(60) Provisional application No. 61/582,260, filed on Dec. 31, 2011.
(60) Provisional application No. 61/530,323, filed on Sep. 1, 2011.

(51) Int. Cl. A01K 67/027; C07K 16/46; C12N 15/85

(52) U.S. Cl. CPC A01K 67/0278; C12N 15/8509; A01K 67/0276; A01K 67/0271; C07K 16/462; A01K 2207/15; A01K 2217/072; A01K 2217/075; A01K 2227/30; A01K 2267/01; C12N 2015/8518; C07K 2317/24; C07K 2317/56; C07K 2317/14; C07K 2317/23; C07K 2317/52; C07K 2317/565

(57) ABSTRACT

The invention relates to transgenic birds capable of producing chimeric immunoglobulins, with a combination of human and avian sequence, in their B cells. In some embodiments, the birds are chickens. When challenged with an antigen, the transgenic avians produce antigen-specific functional antibodies. The invention also relates to light chain immunoglobulin transgenes for making such transgenic avians, as well as methods and vectors for disrupting endogenous immunoglobulin loci in birds.

### RELATED APPLICATIONS

This application claims the benefit of U.S. Provisional Application No. 61/530,323, filed Sep. 1, 2011 and U.S. Provisional Application No. 61/582,260, filed Dec. 31, 2011. The entire teachings of the above applications are incorporated herein by reference.

### STATEMENT OF FEDERAL FUNDING

This invention was made with government support under Cooperative Agreement No. 70NANB7H7003 from the National Institute of Standards and Technology. The government has certain rights in the invention.

### BACKGROUND OF THE INVENTION

Monoclonal antibodies, with their specificity for a single antigenic determinant, have rapidly become adopted as therapeutic agents, with eight such antibodies having sales of more than $1 billion each in 2007. (Scolnik, P., mAbs: A business perspective. mAbs 1(2): 179-184 (2009)). Polyclonal antibodies have the potential to provide even greater therapeutic benefits through their ability to target simultaneously multiple antigenic determinants present on a target. Non-specific polyclonal antibodies produced from pooled human plasma have been demonstrated to have therapeutic benefit for certain conditions such as inflammation and are termed “Intravenous Immunoglobulin” or IVIG and are currently marketed by several companies, including Baxter. Various companies have attempted to further exploit the potential of polyclonal antibodies by focusing on the production of recombinant polyclonal antibodies, typically in cell culture. See, e.g., Wiberg et al., “Production of target-specific recombinant human polyclonal antibodies in mammalian cells,” in Biotechnol. Bioeng. 94(2):396-405 (2006).

Another approach to recombinant polyclonal antibody production would be to produce polyclonal antibodies in animals. Due to their ability to bind a multiplicity of sites on a target antigen, polyclonal antibodies are highly useful as therapeutic agents. Unfortunately, due to their diverse nature, polyclonal antibodies represent natural products that are highly difficult to produce recombinantly compared to monoclonal antibodies. Accordingly, most if not all commercially available polyclonal antibodies for therapeutic use against a specific set of targets are produced by immunizing animals. For example, Anascorp®, approved by the FDA in 2011 as the first specific treatment for scorpion stings, is manufactured from the plasma of horses immunized with venom from four types of scorpions, while Digibind® and DigiFab®, antigen binding fragments of polyclonal antibodies produced in sheep immunized with dixogin, are used to treat persons suffering digoxin overdoses.

Unfortunately, antibodies produced in animals may themselves produce an immune response in humans. For some therapeutic uses, such as the binding of venom or dixogin contemplated by the products noted above, the animal antibodies may be treated with papain or pepsin to produce antigen-binding Fab or F(ab′)₂ fragments free of the Fc portion. In some cases, removing the Fc fragment reduces some of the antibodies' non-human sequence and, it is hoped, some of the immune response, without removing any of the antigen binding properties. Removing the Fc portion of the antibody is, however, not possible for uses in which the Fc portion is needed for the intended therapeutic effect, for example, to induce antibody-dependent cell-mediated cytotoxicity, or “ADCC.”

Another approach to reducing the immunogenicity in humans of antibodies produced in animals is to create transgenic animals that produce antibodies in which some or all of the immunoglobulin sequence native to the animal has been replaced by human or partially humanized sequences. For example, transgenic mice have been created which produce chimeric antibodies in which the mouse Fc region is replaced with a human Fc region. There are several drawbacks to this system including, immunogenicity from other regions of the antibodies and that fact that mice are mammals with low phylogenetic distance from human. The issue most difficult to overcome, however, is that these animals are small and are not capable of producing large quantities of antibodies. Further, obtaining the antibodies requires either bleeding the animals or draining ascites from the abdominal cavity. Other mammals used to raise antibodies, such as rabbits and goats, also require bleeding to obtain antibodies.

An alternative would be to produce polyclonal antibodies in birds. Bird antibodies are present in relatively high levels in eggs, and the localization of antibodies in eggs removes the need to bleed the animal to obtain them. Further, bird husbandry is well understood, as exemplified by the millions of chickens, turkeys, pheasants, ducks, geese, ostriches, and other birds raised worldwide for food and for egg production. The ability to produce antibodies in eggs would make production and isolation of substantial quantities of antibodies available in a particularly convenient form. Protocols are well established to purify antibodies from egg yolks. Moreover, as chickens are not as close to humans genetically or evolutionarily as are other mammals, weak human antigens may be able to trigger a strong immune response in chickens and the consequent generation of high quality polyclonal antibodies. Additionally, the transgenic chicken expression system has substantial advantages over vertebrate, plant, or bacterial cell expression systems, particularly in its ability to provide large quantities of antibody product. Unfortunately, the procedures that work well in mice and perhaps in other mammals to create transgenic animals expressing chimeric or humanized antibodies do not work in birds.

Mammalian antibodies typically comprise two Ig light chains and two Ig heavy chains, which are bound to the light chains and to each other by disulfide bonds. The light and heavy chains comprise variable regions responsible for antigen recognition and binding. Mammals such as humans and mice generate their repertoire of antibodies by a process of Ig gene rearrangement which takes place during B cell maturation in the bone marrow. As described by Goldsby et al., Kuby Immunology, 4^{th} Ed., W.H. Freeman and Co. (New York, 2000), the heavy chain variable regions rearrange first, followed by the light chains. The lambda and kappa light chain families contain V (variable), J (joining), and C (constant) gene segments, the rearranged V and J segments of which combine to encode the variable region portion of the light chain, while the heavy chain family contains V, D (diversity), J, and C gene segments, the V, D, and J segments of which combine to encode the variable regions of the heavy chain. This is termed V(D)J recombination. The lambda chain multigene family in humans, for example, contains some 30 V gene segments, 4 J gene segments, and 4 C gene segments, while the kappa chain multigene family contains some 40 V gene segments, 5 J gene segments, and 1 C gene segment. The heavy chain multigene family in humans comprises some 51 V gene segments, 27 D gene segments, 6 J gene segments and a series of C gene segments. Antibody diversification is achieved by a combination of combinatorial joining of these V, D, and J segments within the respective chains, junctional flexibility, P-region and N-region nucleotide additions, and somatic hypermutation, particularly localized in the complementarity determining regions of the light and heavy chains. See, generally, Goldsby et al., supra.

Antibody production in birds differs in important ways from that of mice and humans One such difference is the manner in which humans and birds generate antibody diversity. The human immune system generates enormous antibody diversity by V(D)J recombination. The avian system is unable to do so. The chicken Ig light chain gene locus, for example, has one V gene segment and one J gene segment, a sharp contrast to the 30 V gene segments and 4 J gene segments available to add diversity to the human Ig light chain. Similarly, the chicken Ig heavy chain has one V gene segment and one J gene segment, a sharp contrast to the 51 V gene segments and 6 J gene segments (plus 27 D gene segments) of the human Ig heavy chain. While chickens thus lack V and J gene diversity and the corresponding ability to generate diversity by rearranging V and J segments, however, they have a large cluster of pseudogenes upstream of the immunoglobulin loci. The V_{L} and V_{H} sequences can be replaced in chickens by pseudogene sequences through a process known as somatic gene conversion, which is not used by humans Somatic gene conversion permits chickens to generate antibodies with a diversity similar to that of humans despite the constraint on V-J rearrangements.

A second, striking difference between antibody production in mammals and birds is where their antibodies undergo maturation. In mammals, antibody maturation occurs in the bone marrow. In birds, somatic gene conversion occurs only in an organ known as the bursa of Fabricius. Further, while mammals are capable of undergoing antibody maturation throughout their life, avian B cells mature only for a period stretching from late embryonic stage through a few weeks after hatching. See, Davison, Kaspers and Schat (eds.), Avian Immunology, Academic Press (San Diego, Calif., 2008), at, e.g., chapters 1, 4 and 6. Moreover, the Fc region of chicken antibodies is needed to drive antibody maturation in the bursa of Fabricius. Therefore, the B-cell development pathway, the immunoglobulin gene rearrangement, and the process of cell maturation and evolved antibody specificities are different in birds than in mammals.

It would be useful to be able to generate antibodies in birds that are of human sequence and therefore more suitable for in vivo use in humans than are conventional avian antibodies. The present invention meets these and other needs.

### BRIEF SUMMARY OF THE INVENTION

In a first group of embodiments, the invention provides recombinant nucleic acid constructs which, when present in an avian B cell, result in expression of an antibody comprising both avian and mammalian elements. In some embodiments, the avian is a Galliformes. In some embodiments, the Galliformes avian is of the species Gallus gallus. In some embodiments, the Gallus gallus is of the subspecies domesticus (e.g., a chicken). In some embodiments, the antibody comprises a chicken immunoglobulin light chain constant region. In some embodiments, the antibody comprises a chicken immunoglobulin light chain variable region. In some embodiments, the antibody comprises a human or humanized immunoglobulin light chain variable region. In some embodiments, the antibody comprises a human or humanized immunoglobulin light chain constant region. In some embodiments, the nucleic acid construct comprises a promoter operative in a chicken B cell and configured to drive expression of the antibody after rearrangement of elements in the construct. In some embodiments, the antibody comprises at least one immunoglobulin chain, which chain comprises a chicken constant region and a human or humanized variable region.

In a further group of embodiments, the invention provides isolated nucleic acid molecules comprising a plurality of human or humanized pseudogenes, wherein each pseudogene is 20 nucleotides to about 1000 nucleotides in length and has sufficient homology to a segment encoding a human or humanized variable (V_{L}) light chain to permit gene conversion when said molecule is present in an avian immunoglobulin light chain locus in an avian B cell during B cell maturation. In some embodiments, the portion of a human or a humanized VL chain comprises a variable light chain framework region. In some embodiments, the portion of a human or a humanized VL chain comprises a variable light chain complementarity determining region. In some embodiments, the portion of a human or a humanized VL chain comprises portions both of a variable light chain framework region and of a complementarity determining region. In some embodiments, the plurality of pseudogenes is between 5 and 95 pseudogenes.

In yet a further group of embodiments, the invention provides isolated nucleic acid constructs comprising, in the following order, read 5′ to 3′: (a) a plurality of human or humanized pseudogenes, wherein the pseudogenes comprise a nucleotide sequence of from 20 nucleotides to about 1000 nucleotides, optionally wherein said sequence encodes at least a portion of a human or a humanized VL chain, (b) a promoter operative in an avian B cell, and (c) a variable region segment encoding a variable region of a human or humanized light chain (HuVL), wherein the promoter is operatively linked with the variable region segment and wherein each pseudogene has sufficient homology to the segment encoding HuVL to permit gene conversion when the construct is present in an avian immunoglobulin light chain locus in an avian B cell during B cell maturation. In some embodiments, the construct further comprises (d) a nucleic acid sequence encoding a human, humanized, or avian constant region. In some embodiments, the avian B cell of step (b) and said avian immunoglobulin light chain locus B cell of step (c) are a chicken B cell. In some embodiments, the HuVL is a human immunoglobulin kappa light chain variable region.

In yet another group of embodiments, the invention provides targeting vectors comprising a nucleic acid construct of any of the preceding claims. In some embodiments, the targeting vectors comprise an attP site.

In another group of embodiments, the invention provides expression cassettes comprising any of the nucleic acid constructs or targeting vectors described above.

The invention also provides insertion vectors which comprise any of the nucleic acid constructs described above, and an insertion sequence permitting insertion into an avian immunoglobulin light chain gene in an avian cell. In some embodiments, the insertion sequence comprises an attP sequence. In some embodiments, the avian immunoglobulin light chain gene and the avian cell are from Gallus gallus domesticus.

In a further group of embodiments, the invention provides recombinant avian chromosomes comprising a first nucleic acid sequence, which sequence comprises, in the following order, read 5′ to 3′: (a) a plurality of human or humanized pseudogenes, wherein said pseudogenes comprise a nucleotide sequence of from 20 nucleotides to about 1000 nucleotides, optionally wherein said sequence encodes some or all of a human or a humanized VL chain, (b) a promoter operative in an avian B cell, and (c) a variable region segment encoding a variable region of a human or humanized light chain (HuVL), wherein the promoter is operatively linked with the variable region segment and wherein each pseudogene has sufficient homology to the segment encoding the HuVL to permit gene conversion when the first nucleic acid sequence is present in an avian immunoglobulin light chain locus in an avian B cell during B cell maturation. In some embodiments, the chromosome further comprises (d) a second nucleic acid sequence encoding a human or an avian constant region. In some embodiments, the avian B cell of step (b) and said avian immunoglobulin light chain locus B cell of step (c) are a chicken B cell.

In a further group of embodiments, the invention provides avian cells comprising any of the nucleic acid constructs described above.

In still a further group of embodiments, the invention provides an avian cell comprising a recombinant avian chromosome, which chromosome comprises a first nucleic acid sequence, which sequence comprises, in the following order, read 5′ to 3′: (a) a plurality of human or humanized pseudogenes, wherein each pseudogene comprises a nucleotide sequence of from 20 to about 1000 nucleotides, optionally wherein said sequence encodes some or all of a human or a humanized VL chain, (b) a promoter operative in an avian B cell, and (c) a variable region segment encoding a variable region of a human or humanized light chain (HuVL), wherein the promoter is operatively linked with the variable region segment and wherein each pseudogene has sufficient homology to the segment encoding the HuVL to permit gene conversion when the first nucleic acid sequence is present in an avian immunoglobulin light chain locus B cell during B cell maturation. In some embodiments, the recombinant avian chromosome further comprises: (d) a second nucleic acid sequence encoding a human or an avian constant region. In some embodiments, the avian is a Galliformes.

In an additional set of embodiments, the invention provides avian cells comprising any of the nucleic acid construct described above, wherein said nucleic acid construct replaces or disrupts expression of at least one endogenous immunoglobulin light chain gene locus.

In still a further group of embodiments, the invention provides birds comprising any of the nucleic acid constructs described above. In some embodiments, the bird is a Galliformes. In some embodiments, the Galliformes is of the species Gallus gallus. In some embodiments, the bird is Gallus gallus domesticus.

In yet a further group of embodiments, the invention provides birds comprising any of the nucleic acid constructs described above, wherein the bird produces antibodies comprising a human or humanized variable light region and an avian, human, or humanized constant region. In some embodiments, the bird is a Galliformes. In some embodiments, the bird is Gallus gallus domesticus (chicken). In some embodiments, the chicken does not produce antibodies comprising both a chicken variable light region and a chicken constant region.

In a further group of embodiments, the invention provides monoclonal antibodies comprising a human or humanized variable light chain region and an avian constant light chain region. In some embodiments, the avian is Gallus gallus domesticus.

In a further group of embodiments, the invention provides compositions of polyclonal antibodies, which antibodies comprising humanized variable regions and avian constant regions. In some embodiments, the avian is Gallus gallus domesticus.

In a further group of embodiments, the invention provides chicken eggs comprising yolk, said egg containing an antibody comprising a human or humanized variable region. In some embodiments, the antibody further comprises a chicken constant region. In some embodiments, the antibody further comprises a human or humanized constant region. In some embodiments, the antibody is present in said yolk of said egg.

In a further group of embodiments, the invention provides methods of making polyclonal antibodies specific for a target antigen. The methods comprise contacting a bird described above with the target antigen. In some embodiments, the bird is Gallus gallus domestics. In some embodiments, the contacting is by injecting the antigen into the bird.

In a further group of embodiments, the invention provides chicken cell lines producing monoclonal antibodies, which antibodies comprise a humanized variable region. In some embodiments, the antibodies have a chicken constant region. In some embodiments, the antibodies have a human or humanized constant region.

In still other embodiments, the invention provides chicken cell lines producing polyclonal antibodies, which antibodies comprise a human or humanized variable region. In some embodiments, the antibodies further comprise a chicken constant region. In some embodiments, the antibodies further comprise a human or humanized constant region.

In a further group of embodiments, the invention provides methods of making a transgenic bird comprising: a) in a primordial germ cell of a bird, knocking out a bird immunoglobulin gene; b) inserting into the knocked out immunoglobulin gene: (i) at least one human or humanized pseudogene, wherein said pseudogene is under control of a promoter operative in a B cell of said avian; (ii) at least one human or humanized immunoglobulin gene segment selected from the group consisting of a Variable immunoglobulin gene segment, and a Joining immunoglobulin gene segment, and (iii) a segment encoding a human or a chicken constant region, thereby creating a transgenic primordial germ cell; c) introducing said transgenic primordial germ cell into a bird embryo; and d) growing said bird embryo into an adult bird such that said transgenic germ cell integrates into a germline of said embryo. In some embodiments, the bird is a Galliformes. In some embodiments, the Gallifomes is of the species Gallus gallus.

In a further group of embodiments, the invention provides methods comprising: a) collecting an egg laid by any of the bird described above, wherein said egg comprises polyclonal antibodies produced by said bird; and b) isolating said polyclonal antibodies.

### INCORPORATION BY REFERENCE

All publications and patent applications mentioned in this specification are herein incorporated by reference to the same extent as if each individual publication or patent application was specifically and individually indicated to be incorporated by reference.

### BRIEF DESCRIPTION OF THE DRAWINGS

The application file contains at least one drawing executed in color. Copies of this patent or patent application publication with color drawing(s) will be provided by the Office upon request and payment of the necessary fee.

FIG. 1 is a photograph of a Southern blot showing the IgL small deletion knockout passed through the germline and bred to homozygosity. Genomic DNA samples were collected from Day 3 embryos from KO7/+ by KO7/+ mating and analyzed by Southern blotting. A mutant band (4.0 Kb) was observed in KO7/KO7 embryos.

FIG. 2 is a photograph showing PCR genotyping of offspring of IgLKO/+ to IgLKO/+ breeding. Genomic DNA samples were collected from combs on the first day after hatching and PCR was performed using CLC2F/CLC1R primers for wild type IgL and ERNI+79F/neo3 primers for ERNI-neo.

FIG. 3 graphs flow cytometry results showing that chIgL KO/KO mutants lack peripheral B lymphocytes. Cells were collected from either the Bursa of Fabricius of newly-born chicks or from the peripheral blood of wild type, IgLKO/+, and IgLKO/KO chickens. Flow cytometric analysis was done following staining of cells with PE-conjugated anti-Bu-1 antibody. Histogram with a percentage shows the portion of Bu-1+ cells in each sample.

FIG. 4A is a schematic diagrams showing a human V insertion vector consisting of four major parts (from left to right): the human pseudogene array, chicken IgL sequences such as the promoter, introns, a human functional V gene, the chicken IgL constant region, an attB site for insertion into the chicken IgL locus, and the β-actin promoter. FIG. 4B shows two versions of a human V insertion vector. The top version, version 1A, does not contain an intron between the leader sequence and the hV sequence, while the second, version 1B has a 125 bp chIgL V leader intron between those sequences.

FIG. 5 sets forth the amino acid sequence of functional huV. The amino acid sequence of functional huVK (SEQ ID NO:37, top) was aligned against the germline configuration of the human VK sequence (SEQ ID NO:38, bottom). Five amino acids are different between the sequences, at the positions marked by the asterisks.

FIG. 6 shows a huVK Pseudogene alignment. Individual huVK pseudogenes were translated into protein sequences and alignment analysis was performed against the germline configuration of human VK pseudogene. Amino acids that are different are highlighted.

FIG. 7 is a schematic diagram of huVK insertion in small deletion IgL knockout in DT40 cells. The figure shows a chicken IgL locus containing three pseudogenes (YV3, YV2 and YV1), a promoter, a V region, a J region and a C region. Below is the IgL targeting vector (IgL pKOSD). IgL pKOSD contains a β-actin-neo cassette, a promoterless puromycin and an attP site at the 5′ end of the puro gene. It was designed to target a small region of the genome (about 3 kb) including the chicken IgL J and C regions. Below shows the integrated targeting vector. The huVK insertion vector contains a β-actin promoter, human VK pseudogenes, chicken IgL promoter, human VK functional gene, chicken J-C intron, chicken C region and an attB site. There is also a β-actin-EGFP gene for visualization of cells by green fluorescence. The lower panel illustrates integrase-mediated insertion in the presence of phiC31 integrase.

FIG. 8 is a schematic diagram of human VK insertion into small deletion IgL knockout in PGCs (KO-07). The figure shows the genome structure of a targeted chicken IgL locus by an IgL targeting vector (IgL pKOSB). IgL pKOSB contains a pErni-neo cassette, a promoterless puromycin and an attP site at the 5′ end of the puro gene. The huVK insertion vector contains a β-actin promoter, human VK pseudogenes, chicken IgL promoter, human VK functional gene, chicken J-C intron, chicken C region and an attB site. The lower panel illustrates integrase-mediated insertion of human IgL pseudogene array and human VK into targeted chicken IgL locus in the presence of phiC31 integrase.

FIG. 9 is a schematic diagram of huVK insertion in large deletion IgL knockout in PGC cells (KO-12 & KO-13). The figure shows genome structure of targeted chicken IgL locus by IgL targeting vector (IgL pKO7C). IgL pKO7C contains a β-actin-neo cassette, a promoterless puromycin and an attP site at the 5′ end of the puro gene. It was designed to target a large region of the genome (about 27 kb) including the chicken IgL array, V, J and C regions. Below depicts the structure of huVK insertion vector. It contains a β-actin promoter, human VK pseudogenes, chicken IgL promoter, human VK functional gene, chicken J-C intron, chicken C region and an attB site. There is also a β-actin-EGFP gene for visualization of cells by green fluorescence. A HS4 insulator sequence and loxP sites are also included. The lower panel illustrates integrase-mediated insertion of human IgL pseudogene array and human VK into targeted chicken IgL locus in the presence of phiC31 integrase.

FIG. 10 is a schematic diagram of a strategy for PCR genotyping of KI7B chickens. Primers were designed to amplify the Erni-neo cassette, huVK knockin elements, and wild-type chicken IgL locus, respectively. Arrows indicate the approximate location of the primers.

FIG. 11A is a gel showing PCR genotyping results of KI7B chickens. Primers were used for detection of WT chIgL and ERNI-neo. WT chIgL produced a 2.2 kb fragment while ERNI-neo produced a 750 bp fragment. Genomic DNA was prepared from the combs of newly-born chicks. FIG. 11B a gel showing PCR genotyping results of KI7B chickens. Primers were used for detection of huVK knockin. huVK knockin showed a 600 bp fragment. Genomic DNA was prepared from the combs of newly-born chicks. FIG. 11C is a gel showing PCR results of KI7B chickens. PCR using primers specific for female W-chromosome was also performed for sex determination of the birds and PCR for actin was used as control.

FIG. 12 is a diagrammatic presentation of the outcomes of human VK insertion into IgL KO DT40 cells. Insertion of huVL into IgL KO DT40 cells could have 2 different outcomes depending on which allele of the IgL locus was knocked out. Left panel: If the rearranged allele was knocked out, insertion of the huVK would restore the expression of sIgM. Right panel: if the knockout allele was not rearranged, insertion of huVK could result in expression of chimeric sIgM.

FIG. 13 presents photographs of gels showing chimeric IgL mRNA expression in IgL KI DT40 cells. IgL KO DT40 cells were transfected with HuVK insertion vector and puromycin-resistant clones were selected. After preparation of messenger RNA, RT-PCR was performed using one primer specific for huVK and the other for chicken IgL C region. Top panel: detection of an approximately 400 bp PCR product indicated expression of chimeric IgL mRNA.

FIG. 14 is a photograph of a gel showing detection of chimeric IgL protein expression in DT40 cells. Western analysis was performed on puromycin-resistant clones after transfection of IgL KO DT40 cells with huVK insertion vector. Wild type (WT) DT40 cells, IgL KO DT40 cells, and human B cells were used as control. Anti-chIgY (H+L) antibody was able to detect both IgY heavy (approximately 32 kD) and light chain (approximately 25 kD) in WT DT40 cells, but only light chain in chIgL-huV 1B cells. Neither heavy nor light chain was detected in IgL KO DT40 cells.

FIG. 15 presents scatter plots showing detection of chimeric IgL on the surface of DT40 cells by flow cytometry. DT40 WT DT40 cells carrying chIgL-huV gene along with wild-type (WT) and chIgL KO DT40 cells and human B cells were stained for chicken IgM (chIgM), chicken IgY (chIgY) (H+L), and human IgK (huIgK). chIgL-huV DT40 cells were positive for chIgM, chIgY, and huIgK, suggesting these cells expressed chimeric IgL that traffics to cell surface.

FIG. 16 is a diagram showing a strategy for Southern analysis of chIgL-huV. The upper and middle panels show the genomic structure after insertion of huVK into chicken IgL locus with either a small (upper) or large (middle) deletion. The lower panel shows the genomic structure of the WT allele of chIgL locus. A genomic segment upstream of chicken VX was chosen as a probe. The expected genomic fragments detected by Southern analysis are indicated for each genotype.

FIG. 17 is a photograph of a gel showing Southern analysis of huVK knockin PGC clones. Three PGC clones with either small (KO-07) or large (KO-12 and KO-13) deletion in IgL locus were transfected with HuVK insertion vector and puromycin-resistant clones were selected for Southern analysis. A 0.5 kb SacI-BstEII fragment from 10 kb SacI clone upstream of chIgL V was used as a probe. Three fragments (3.9, 8.2, and 10.1 kb) were detected in KO-07 transfected with Seq1A and Seq1B (KO-07/Seq1A and KO-07/Seq1B) while 2 fragments (8.2, and 10.1 kb) were detected in KO-12 and KO-13 transfectants, indicating successful integration of huVK vector into targeted chIgL locus.

FIG. 18 presents photographs of gels showing PCR genotyping of offspring of IgLKI/+ to IgLKI/+ breeding. Genomic DNA samples were collected from combs on the first day after hatching and PCR was performed using CLC2F/CLC1R primers for wild type IgL, ERNI+79F/neo3 primers for ERNI-neo, and huVnd CLC1R for huVK insertion.

FIG. 19 present scatter plots showing B cells produced in chickens of various genotypes. Blood samples were collected from IgL KO/KO, IgL KO/KI and WT barred rock chickens and lymphocytes were isolated and stained for B cells (CD3- and Bu-1+). Results suggested that insertion and expression of huVK into KO IgL locus restored B cell population.

FIG. 20 presents photographs of gels showing chimeric IgL mRNA expression in IgL huVK KI B cells. Lymphocytes were collected from peripheral blood of IgL huVK KI chickens. After preparation of messenger RNA, RT-PCR was performed using primers specific for: huVK (huVK3-20sig-F) (top panel), chicken IgL C region (middle panel), as described in Example 26, or actin (bottom panel). huVK-specific PCR product indicated expression of chimeric IgL mRNA.

FIG. 21 shows the expression of chimeric human k chain in B cells of IgL KI7B chickens. Lymphocytes were isolated from 3 IgLKI/KO and 2 WT barred rock chickens and stained for human k light chain. The left panel presents histograms showing significant shifts of fluorescence intensity to the right in KI7B preps, suggesting expression of human K chain. Legend: Wild type (WT) chicken 1, thin solid line. WT chicken 2, thick solid line. Chicken KI7B-4, thick dashes. Chicken KI7B-21, thin dashes. Chicken KI7B-5, dots. The right panel is a table showing the kappa positive gate and the percentages of kappa positive cells for each chicken.

FIG. 22 shows YVK alignment and gene conversion frequency. B cells from peripheral blood of KI7B chickens were isolated and mRNA was prepared, followed by RT-PCR and TA-cloning. Sequencing analysis showed that gene conversion had modified the functional human V, using donor sequences from the upstream human pseudogene pool.

FIG. 23 shows five examples of gene conversion in the chimeric light chain V region.

FIG. 24 is a schematic diagram of immunization procedure. Three chickens of each of 3 different genotypes (WT, KI/KO, KO/KO) were chosen for immunization with tetanus toxoid vaccine (for cattle). Time points for immunization and blood collection were labeled.

FIG. 25 is a graph showing antigen-specific antibodies in chimeric chickens. Serum was collected after immunization and analyzed using an ELISA kit for tetanus-specific antibodies. Both IgL KO/KI and WT barred rock chickens produced significant amount of anti-tetanus antibodies, while KO/KO chickens did not. The amount of antibodies is expressed as International Units (IU) (n=3). Legend: Line connecting diamonds: Wild type (WT). Line connecting filled in squares: KO/KO. Line connecting triangles: KI/KO.

### DETAILED DESCRIPTION

Polyclonal antibodies are highly useful as therapeutic agents, but may be difficult to produce recombinantly. Monoclonal and polyclonal antibodies can be produced in non-human mammals, but that requires bleeding the animals and isolating the antibodies from the serum. Generating antibodies in birds that produce the antibodies in their eggs obviates the need to bleed the animals to obtain the antibodies. Unfortunately, bird antibodies induce immune responses in humans, and the techniques used in mammals to create transgenic animals that produce chimeric or humanized antibodies with reduced immunogenicity do not work in birds.

Surprisingly, the present invention solves these problems. The studies reported in the Examples demonstrate the production of transgenic birds that produce chimeric antibodies with at least one variable chain in which endogenous bird sequences have been “knocked-out” and replaced by human variable chain gene sequences. Female transgenic birds produced in the course of the studies reported herein produced chimeric antibodies. Such antibodies can be isolated by bleeding the transgenic animals or by collecting antibodies incorporated into eggs. Further, the studies reported herein demonstrate that transgenic birds immunized with an exemplar antigen, tetanus toxoid, produced polyclonal chimeric antibodies specifically binding the antigen. Accordingly, the polyclonal chimeric antibodies produced by the transgenic birds were fully functional. Moreover, the birds produced the chimeric antibodies in amounts similar to the amount of polyclonal antibodies produced against the same antigen by immunized wild type birds. Both results are particularly striking given the differences between the methods in which humans and birds generate antibody diversity.

U.S. patent application Ser. Nos. 12/896,681 and 11/977,538 teach the disruption of endogenous immunoglobulin genes in chicken embryonic stem cells, resulting in the production of chimeric chickens in which endogenous immunoglobulin production has been “knocked-out.” U.S. patent application Ser. Nos. 11/977,538 and 10/104,486 describe vectors suitable for performing such “knock-outs.”

The studies reported herein demonstrate that transgenic birds can be produced in which the endogenous genes encoding avian antibody variable light domains (“VL”) can be deleted (or “knocked out,” (sometimes herein referred to as “KO”)), and that sequences encoding an engineered chimeric antibody light chain comprising an avian constant region and a variable region based on a human antibody gene can be inserted, or “knocked in” (sometimes herein referred to as “KI”).

Gene conversion is an intrachromosomal process used in birds to generation antibody diversity. The process uses upstream pseudo-IGVL genes as donor sequences and is described in, e.g., McCormack W T and Thompson C B, “Chicken IgL variable region gene conversions display pseudogene donor preference and 5′ to 3′ polarity,” Genes Dev. 4:548-558 (1990) and McCormack W T, et al., “Avian B-cell development: generation of an immunoglobulin repertoire by gene conversion,” Annu Rev Immunol. 9:219-241 (1991).

Surprisingly, despite the fact that antibody diversity in humans is generated by V(D)J recombination, while antibody diversity in birds is generated by gene conversion, the studies herein show the introduced human sequence underwent gene conversion in the transgenic birds. Because the chimeric variable light chain included bird constant regions but human variable regions, it was unknown whether the chimeric light chain could pair with endogenous bird heavy chain to form antibodies capable of antigen binding. The results of the antibody staining studies reported herein indicated that the chimeric light chain protein binds antigen with the avian heavy chain, indicating that it folded properly and trafficked appropriately within the cell.

The generation of functional chimeric antibodies with a human variable region is further surprising given that mammalian antibodies mature in the bone marrow and those of birds mature in the bursa of Fabricius, an organ that does not have a mammalian counterpart. The studies herein, however, show that exemplar transgenic birds, transgenic chickens, were created in which the endogenous genetic locus encoding chicken VL chains were knocked out and in which a construct of genetic information derived from an exemplar human variable light chain, the kappa (κ) chain (human kappa variable light chains will sometimes be referred to herein as “VK” or “huVK” chains), was introduced, or “knocked in.”

The transgenic birds were then challenged by immunization with an exemplar antigen, tetanus toxoid vaccine. As reported in the Examples, below, the transgenic chickens were hyperimmunized with tetanus toxoid vaccine. Blood samples were collected and the amount of tetanus specific antibody was analyzed. The results reported in the Examples show that functional tetanus specific antibodies were present in the serum of transgenic chickens carrying the chimeric light chain. Further, although the levels of the antibodies produced by the KI/KO chickens were lower than in wild type chickens during the first three weeks, by four weeks, the levels were similar. As expected, since KO/KO chickens do not produce B cells, KO/KO birds were unable to produce specific antibodies. This demonstrates that the human variable light kappa chain used as an exemplar chain, could serve as a component of a functional and antigen specific B cell receptor and that insertion of huVK into a partially deleted IgL locus of KO/KO chickens restored B cell development and the ability of these chickens to produce antigen-specific antibody following hyperimmunization. Given that the human kappa light chain isotype has a more complicated genomic organization than does the human lambda light chain, it is expected that the results achieved using the human kappa chain sequence would be obtained using the human lambda light chain as well.

A further surprising aspect of the present invention is that human pseudogenes derived from a human VK expressed sequence tag database functioned to provide diversity to the antibodies produced by transgenic birds bearing light chain variable regions into which human pseudogenes had been inserted. As previously noted, birds produce antibody diversity through an interchromosomal gene recombination process known as gene conversion, a process which does not occur in humans During gene conversion, antibody diversity is generated by a process in which portions of pseudogene sequences replace homologous portions of the V chain-encoding sequence. The section recombined may then itself undergo recombination with the sequence of yet another pseudogene. These iterative recombination events result in the ability of the avian immune system to produce a large number of diverse antibodies. It was unknown if the regulatory elements in a bird genome and the avian enzymes that mediate gene conversion in an avian B cell or B cell precursor would work with pseudogenes derived from human variable light chain sequences and recombine them with a human variable chain gene locus inserted within an avian immunoglobulin gene locus. Surprisingly, the studies reported here show they did. It was further unknown whether, if so, the host avian system would be able to use these introduced human sequences to form antigen-specific functional antibodies. Surprisingly, the studies reported here show they did.

The studies set forth below report the insertion of human variable region sequences into two chicken cell types (these chicken cells carrying chimeric chicken-human sequences are sometimes called “chIgL-huV” herein to indicate that the chicken light chain locus has been modified with the human V region). To produce transgenic chickens carrying the chIgL-huV knockin, chIgL-huV primordial germ cells (PGCs) were injected into the bloodstream of Stage 14-16 embryos. The embryos were grown, chicks (G0) hatched, and the G0 potential germline chimeric males were grown to sexual maturity. The G0 males were test mated to wild type Barred Rock hens by artificial insemination to pass the genetic modification on to the next generation (G1) and produce fully transgenic chickens carrying the chimeric light chain construct in every nucleated cell of the body.

While the transgenic chickens reported herein had only light chains with a human variable sequence, it can be expected that the antibodies will provoke a reduced non-specific immune response when introduced into humans as compared to antibodies in which both chains are comprised wholly of chicken sequences. Alternatively, individual antibodies with desired properties can be sequenced, and the chicken-derived variable regions from these antibodies can be combined with human-derived constant regions to produce chimeric monoclonal antibodies, or the complementarity-determining regions (CDRs) can be grafted into human antibody framework sequences to produce humanized antibodies. Thus, the ability to create transgenic chickens producing polyclonal antibodies in which the variable region of the light chain has human sequences represents an important advance.

Birds in which Antibodies can be Produced

The birds employed in the exemplar studies reported herein were chickens. Most research on avian immunology has been conducted using the domestic chicken, Gallus gallus domesticus. Davison, Kaspers, and Schat, Avian Immunology, Academic Press, San Diego Calif. (2008) (hereafter, “Davison et al.”), page 1. The avian immune system is, however, organized similarly in all birds and gene conversion, the system by which chickens introduce variability into their antibodies, is a shared feature among avians. For example, Davison et al. note that, while chickens have only one functional VL and other species such as ducks have up to four, gene conversion is still the dominant method of generating diversity. Id., page 5, see also Starck and Ricklefs, Avian Growth and Development, Oxford University Press, Inc., New York (1998), page 206. Chickens and ducks have also been reported to have only one immunoglobulin light chain isotype, lambda, in distinction to reptiles and most mammals, which typically have at least two isotypes, designated as kappa and lambda (frogs also have a third isotype, known as sigma). A recent study regarding the zebra finch, a member of the Order Passeriformes, far removed taxonomically from chickens and ducks, reported that the genomic organization of the light chain locus of the zebra finch was very similar to that of the chicken and that, like chickens and ducks, the zebra finch had only the lambda light chain isotype. Das, S., et al., Mol. Biol. Evol. 27(1):113-120 (2010). Das et al. suggest that the similar genomic organization, with a single functional IGVL followed by multiple IGVL pseudogenes, makes it likely that the zebra finch, like the chicken, generates antibody diversity by intrachromosomal gene conversion, which uses the upstream pseudo-IGVL genes as donor sequences. Das et al., at 118.

Given the common structural genetic features of the bird immune system and the shared method of generating antibody diversity, it is believed that the results shown in the studies reported herein will also obtain in birds generally. In some embodiments, the birds are members of the order Galliformes. In some embodiments, the birds are not anseriforms, which have some features in their immune system different from those of other avian orders. See, e.g., Starck and Ricklefs, supra. In some embodiments, the Galliformes bird is a turkey, grouse, New or Old World quail, ptarmigan, partridge, or pheasant. As persons of skill are aware, the domestic chicken is a subspecies of the Red Junglefowl, Gallus gallus (sometime abbreviated “G.g.”), a member of the Pheasant family, and that a number of other subspecies, such as G. g. murghi India exist. As the Red Junglefowl and the various Gallus gallus subspecies are all members of one species, they can be expected to have identical organization and regulation of the immune system. Accordingly, Red Junglefowl, the various Gallus gallus subspecies and, in particular, G. g. domesticus, are preferred. For purposes of this disclosure, the terms G. g. domesticus and “chicken” are interchangeable.

### Considerations in Producing Polyclonal Antibodies in Birds

In other species, transgenic animals are created by making genetic modifications in embryonic stem cells. A transgene containing DNA that encodes an exogenous product, such as a protein or an antibody, is engineered to incorporate into the genome. These cells have the ability to contribute to the tissue of an animal born from the recipient embryo and to contribute to the genome of a transgenic offspring of a resulting animal. The transgene contains the blueprint for the production of the protein or antibody and contains sufficient coding and regulatory elements to enable the expression of the protein in the animal created from the insertion of the stem cells into a recipient embryo. Thus far, an avian embryonic stem cell encoding recombinant DNA sequences have not produced a transgenic animal that could then pass these gene alterations onto offspring. In chickens, transgenesis has been achieved with primordial germ cells.

Chicken primordial germ cells (PGCs) can pass transgenes to offspring and have been genetically modified using a retroviral vector within a few hours following isolation from Stage 11-15 embryos (Vick et al., Proc. R. Soc. Lond. B 251, 179-182 (1993)). The resulting modification, though, is randomly integrated and the size of the transgene is generally limited to less than about 15 kb, and most commonly less than 8 kb. Site-specific changes to the genome cannot be created using this technology, nor can transferred cells be selected to identify site specific modifications to the exclusion of random integration. The present invention enables stable genetic modifications requiring the insertion of greater than 15 kb, greater than 50 kb or greater than 100 kb of exogenous DNA into the genome of cultured avian PGCs.

Polyclonal antibodies by their nature are a diverse repertoire of immunoglobulins, and can only be expressed by immunoglobulin loci after a series of immunological and molecular events in B cells prior to and upon immunization with specific antigens. Further, for high level production of polyclonal antibodies, the endogenous immunoglobulin loci should be inactivated, as endogenous genes have been reported in several species to interfere with the production of proteins expressed by a transgene. In mice transgenic for human immunoglobulin, disruption of endogenous murine immunoglobulin genes by gene targeting resulted in a significant increase in production of hIgG. The generation of transchromosomic cattle carrying a human artificial chromosome (HAC) vector comprising the entire, germline-configured, hIGH and hIGL chain loci was also reported to produce a very low level of hIgG in their plasma although human immunoglobulin gene rearrangement appeared normal in the cattle. Therefore, inactivation of one or more endogenous genes could enhance production of proteins designed to be expressed in a transgenic animal. In these cases, site-specific recombination is used to inactivate a gene in discrete cells and/or at discrete times during development within the context of an otherwise normal animal development.

The present invention provides transgenic chickens in which the endogenous chicken immunoglobulin light chain genes have been “knocked-out” following the procedures disclosed in, e.g., U.S. patent application Ser. Nos. 12/896,681 and 11/977,538. In addition to utilizing the technologies and reagents provided by these applications, the present invention surprisingly demonstrates successful approaches to humanization of chicken immunoglobulin loci, successful production of transgenic birds carrying the chimeric VL construct, generation of a diverse repertoire of human VK sequences, efficient pairing of human chimeric light chain and chicken heavy chain, and production of antigen-specific chimeric human polyclonal antibodies.

One strategy for producing a bird, such as a chicken, that produces human or humanized antibodies includes knocking out the endogenous avian immunoglobulin gene locus to inactivate the locus, verified by a lack of B cells. This can include using a targeting vector to disrupt the immunoglobulin gene locus, for example, resulting in the removal of endogenous promoter, V region, J region and constant region sequences. A recombinant nucleic acid construct containing elements to produce a humanized immunoglobulin chain can be inserted into the knocked-out locus. Inserting the recombinant nucleic acid in this locus can take advantage of existing regulatory sequences that may aid in creation of a mature B cell encoding a human or humanized immunoglobulin chain. Accordingly, one strategy involves creation of an artificial bird immunoglobulin gene locus comprising pseudogenes containing human or humanized V and/or J regions, a human or humanized variable region sequence downstream and operatively linked to a bird promoter, and a sequence encoding a constant region of choice.

The recombinant nucleic acid construct can comprise nucleic acid sequences from a bird, e.g., from an immunoglobulin gene locus, into which operative elements have been inserted. Operative elements include, from 5′ to 3′, in array of human variable region pseudogenes, a promoter operative in a bird cell to drive expression of an immunoglobulin sequence, a human or humanized sequence encoding a variable chain region and a sequence encoding a bird or a human or humanized constant chain region. A bird J-C intron sequence can be positioned between the sequences encoding the variable region and the constant region.

The sequence encoding the variable region that is positioned downstream of the promoter can be a sequence encoding a mature human or humanized variable region of a mature immunoglobulin including, for example, V and J sequences. The human or humanized pseudogenes have sequences of sufficient homology with the variable region to undergo gene conversion during B cell maturation. Typically within the sequences of homology, the pseudogenes contain nucleotide sequences that act as sequence donors permitting diversity to be generated. These “sequence donor” sequences can be, for example, between 20 and about 1000 nucleotides, more preferably between 30 and about 750 nucleotides, still more preferably between 30 and about 450 nucleotides, and may be even more preferably between 50 to about 350 nucleotides. The sequence donor sequences may be of essentially random sequence, as the recombination events of gene conversion will result in generating antibody diversity. Optionally, the sequence donor nucleotide sequences (e.g., the 20 to about 1000 nucleotide sequence noted above) may encode some or all of a VL chain, typically including variable region sequences, but may also include J region sequences. In some embodiments, the sequence donor sequences may encode CDR and framework region sequences. Sequence homology sufficient for recombination during gene conversion is at least 50%, 75%, 80%, 85%, 86%, 87%, 88%, 89%, 90%, 91%, 92%, 93% 94%, 95%, 96%, 97%, 98%, or 99%, with each successive higher percentage of homology being preferred to the one below it. The pseudogenes in the array are typically separated by spacers, such as by sequences that separate pseudogenes in the endogenous avian locus.

A recombinant nucleic acid construct comprising a humanized bird immunoglobulin gene locus can include an insertion sequence selected to recombine within a knocked out bird immunoglobulin gene locus to allow insertion of this insertion vector into the endogenous bird locus.

B cell precursors in a transgenic bird comprising the artificial immunoglobulin gene locus will, upon maturation, undergo gene conversion in which the human or humanized pseudogenes replace sequence in human or humanized variable region locus resulting, ultimately, in a gene encoding an immunoglobulin chain. Because each B cell precursor undergoes a different gene rearrangement, antibody diversity is created. Upon challenge with an immunogen, B cells that produce antibodies that bind to the immunogen will undergo clonal expansion and will produce polyclonal antibodies against the immunogen.

### DEFINITIONS AND ABBREVIATIONS

Units, prefixes, and symbols are denoted in their Systeme International de Unites (SI) accepted form. Numeric ranges are inclusive of the numbers defining the range. Unless otherwise indicated, nucleic acids are written left to right in 5′ to 3′ orientation. The headings provided herein are not limitations of the various aspects or embodiments of the invention, which can be had by reference to the specification as a whole. Definitions of common terms in molecular biology may be found in Krebs et al., eds., Lewin's Genes X (Jones and Bartlett Publishers, Sudbury, M A, 2011), while information on the immune system in birds is set forth in Davison, supra. The terms defined immediately below are more fully defined by reference to the specification in its entirety.

As used in this disclosure, “antibody” includes reference to an immunoglobulin molecule immunologically reactive with a particular antigen and binding fragments thereof. The term also includes genetically engineered forms such as the exemplar chimeric antibodies discussed herein. For convenience of reference, the term “antibody” is also sometimes used herein to refer to antigen binding fragments of antibodies (e.g., Fab′, F(ab′)₂, Fab, and Fv, unless otherwise required by context. See, e.g., Abbas and Lichtman, Basic Immunology: Functions and Disorders of the Immune System, 3^{rd} Ed., Saunders Elsevier, New York (2011), Kindt et al., eds., Kuby Immunology, 6th Ed., W.H. Freeman & Co., New York (2006).

An antibody immunologically reactive with a particular antigen can be generated by recombinant methods such as selection of libraries of recombinant antibodies in phage or similar vectors, see, e.g., Huse, et al., Science 246:1275-1281 (1989); Ward, et al., Nature 341:544-546 (1989); and Vaughan, et al., Nature Biotech. 14:309-314 (1996), or by immunizing an animal with the antigen or with DNA encoding the antigen.

Typically, an immunoglobulin has a heavy and light chain. In naturally produced immunoglobulins, the chains are connected by disulfide bonds. Each heavy and light chain contains a constant region and a variable region, also known as the constant domain and the light domain. In humans, there are two types of light chain, the kappa, or κ, chain, and the lamda, or λ, chain. Humans have five main heavy chain classes (or isotypes) which determine the functional activity of an antibody molecule: IgM, IgD, IgG, IgA and IgE.

The major immunoglobulin class in birds (as well as in reptiles, amphibia and lungfish) is immunoglobulin Y, or “IgY” and is considered an ancestor of both mammalian IgG and IgE. See, e.g., Taylor et al., Biochemistry, 48 (3):558-562 (2009). Like human IgG, IgY has two heavy chains and two light chains. IgY, however, has a higher molecular weight than that of human IgG due to the presence of an extra heavy chain constant domain and does not have a defined hinge region. The anseriform birds produce a IgY(AFc) that has a molecular weight lower than that of IgG. The avian immunoglobulin now called IgY was called IgG from its discovery in the 1890's until it was renamed around 1970.

Light and heavy chain variable regions contain a framework region interrupted by three hypervariable regions, also called “complementarity-determining regions” or “CDRs.” The extent of the framework region and CDRs have been defined. Kabat et al., Sequences of proteins of Immunological Interest, U.S. Department of Health and Human Services, 1991, which is incorporated here by reference). The Kabat database is now maintained online. The sequences of the framework regions of different light or heavy chains are relatively conserved within a species. The framework region of an antibody, that is the combined framework regions of the constituent light and heavy chains, serves to position and align the CDRs in three dimensional space.

The CDRs are primarily responsible for binding to an epitope of an antigen. The CDRs of each chain are typically referred to as CDR1, CDR2, and CDR3, numbered sequentially starting from the N-terminus, and are also typically identified by the chain in which the particular CDR is located. Thus, a VH CDR3 is located in the variable domain of the heavy chain of the antibody in which it is found, whereas a VL CDR1 is the CDR1 from the variable domain of the light chain of the antibody in which it is found.

A “monoclonal antibody” is an antibody produced by a single clone of B-lymphocytes or by a cell into which the light and heavy chain genes of a single antibody have been transfected. Monoclonal antibodies are produced by methods known to those of skill in the art, for instance by making hybrid antibody-forming cells from a fusion of myeloma cells with immune spleen cells. These fused cells and their progeny are termed “hybridomas.” “Monoclonal antibodies” include humanized monoclonal antibodies.

As used herein, “chimeric antibody” refers to an antibody which includes sequences derived from two different species. Most typically, as used herein, chimeric antibodies include human and bird antibody domains, generally human variable regions and bird constant regions. It may, however, also refer to an antibody with an avian heavy chain and a light chain composed of human variable and constant regions. Which meaning is intended will be clear in context.

A “parental antibody” refers to an antibody, such as one produced by a non-human animal, which is chosen for engineering to improve one or more selected characteristics, such as to reduce its immunogenicity when introduced into a human.

“Humanization,” with reference to an antibody derived from a non-human animal, refers to a process in which residues at particular locations in the non-human antibody are replaced with residues more frequently found at that location in human antibodies, or in which portions of the non-human antibody, such as a framework region or a constant region, are replaced by corresponding regions from a human antibody or antibody library. Humanized antibodies contain, for example, amino acid sequences that are unambiguously human in origin. Humanization of an antibody residue can be performed by, for example, site specific mutagenesis. Humanization of framework or constant regions, or both, can be accomplished by a variety of techniques, including synthesizing a combinatorial library comprising the six CDRs of the non-human target antibody fused in frame to a pool of individual human frameworks. A human framework library that contains genes representative of all known heavy and light chain human germline genes can be utilized. The resulting combinatorial libraries can then be screened for binding to antigens of interest. This approach can allow for the selection of the most favorable combinations of fully human frameworks in terms of maintaining the binding activity of the parental antibody.

Epitopes include antigenic determinants. These are particular chemical groups or peptide sequences on a molecule that are antigenic, i.e. that elicit a specific immune response. An antibody specifically binds a particular antigenic epitope on a polypeptide.

A “promoter” is a nucleic acid sequence sufficient to direct transcription. Also included are those promoter elements which are sufficient to render promoter-dependent gene expression controllable for cell-type specific, tissue-specific, or inducible by external signals or agents; such elements may be located in the 5′ or 3′ regions of the gene. Both constitutive and inducible promoters are included (see e.g., Bitter et al., Methods in Enzymology 153:516-544 (1987)).

“Hyperimmunization” is a heightened state of immunity that is induced by the administration of repeated doses of antigen. It is often used in biotech industry to generate highly active, antigen-specific antibodies in animals.

Immune response includes responses of a cell of the immune system, such as a B cell, T cell, or monocyte, to a stimulus. In one embodiment, the response is specific for a particular antigen (an “antigen-specific response”). In one embodiment, an immune response is a T cell response, such as a CD4+ response or a CD8+ response. In another embodiment, the response is a B cell response, and results in the production of specific antibodies.

Selected abbreviations used in this specification:

“GC” is an abbreviation for “gene conversion.”

“KO” is an abbreviation for “knock out”.

“KI” is an abbreviation for “knock in.”

“V” is an abbreviation for an “immunoglobulin variable region”.

“VL” and “V_{L}” are abbreviations for the variable domain of an immunoglobulin light chain.

“CL” and “C_{L}” are abbreviations for the constant domain of an immunoglobulin light chain.

“VH” and “V_{H}” are abbreviations for the variable domain of an immunoglobulin heavy chain.

“IgL” refers to an avian IgL locus and in the Examples refers to a chicken IgL locus. The structure of the chicken IgL is described in, e.g., U.S. patent application Ser. No. 12/896,681 (the “'681 application”), published as U.S. Published Patent Application 20110023160, at paragraph [0031].

“IgH” refers to is an avian IgH locus and in the Examples refers to a chicken IgH locus. The structure of the chicken IgH locus is described in, e.g., the '681 application at paragraph [0032].

“IgK” refers to the human immunoglobulin kappa light chain, or to nucleic acid sequences encoding such a chain, as required by context.

“VK” and “Vκ” refer to the variable region of a human immunoglobulin kappa light chain.

As described in the '681 application, the chicken IgL locus encodes a single functional V_{L}, gene segment separated by 1.8 kb from a single functional J_{L}, gene segment. A single C_{L} region is located 2 kb 3′ from the J_{L} segment. The functional V_{L} segment, designated V_{L1}, is split in the leader region by a 125-bp intron, and the promoter region of V_{L1} includes a conserved octomer box 32 bp upstream from the TATA box. In a 22-kb region upstream of V_{L1} there are 25 V_{L}-homologous gene segments situated in both transcriptional orientations. All 25 of these V_{L} gene segments are truncated at the 5′ end and lack a leader exon and a promoter region. In addition, most, but not all lack a functional recombination signal sequence (heptamer-spacer-nonamer) at the 3′ end and are not capable of V-J rearrangement. These 25 gene segments are designated as V_{L} pseudogenes, ψV_{L} 1-25.

The chicken IgH locus is also restricted in its capacity for combinatorial diversity (see, Reynaud et al., Cell, 59:171-183 (1989), and Reynaud et al., Eur. J. Immunol. 21:2661-2670 (1991)). The chicken IgH locus consists of a single functional V_{H1} segment located 15 kb 5′ from a single functional J_{H} gene segment, with approximately sixteen D_{H} segments between V_{H1} and J_{H}. There is little sequence variation between germline D_{H} segments, thereby limiting combinatorial diversity. A cluster of 80-100 V_{H} pseudogene segments (ψV_{H}), spanning a region of 60-80 kb, is present 5′ of the functional V_{H1} gene. As in the case of the ψV_{L} segments, the ψV_{H} segments lack a promoter region, leader exon, and recombination signal sequences. Many of the ψV_{H} segments are situated with alternating transcriptional orientation.

Homologous recombination of the knock-out construct with the endogenous locus yields a locus in which the J region is absent. The absence of the J region prohibits V-D-J recombination and therefore, a rearranged immunoglobulin locus cannot be generated and a functional immunoglobulin cannot be expressed, as demonstrated by a lack of B cells.

As noted in the '681 application, when the immunoglobulin gene knockout is achieved with an engineered chromosome, the engineering of the chromosome is preferably performed in a recombination-proficient cell prior to insertion in a pluripotent cell, such as an embryonic stem cell, which is then used to create transgenic animals. Gene targeting and recombination in embryonic stem cells has limitations. Certain cell types have been isolated that are recombination proficient. One example is the avian pre-B cell line, commonly designated DT40. Recombination proficient cell lines which display an enhanced frequency of homologous recombination with targeting constructs preferably feature at least two regions of homology flanking a selectable marker. A preferred recombination proficient cell line is the avian DT40 pre-B cell, described in U.S. Pat. No. 5,543,319. See also, e.g., Winding and Berchtold, J Immunological Meth, 249(1-2):1-16 (2001). Cells with increased rates of homologous recombination may be identified by known techniques (see, e.g., Buerstedde and Takeda, Cell 67:179-188 (1991)). DT40 cells are highly efficient in gene targeting recombination events and have been used to modify mammalian genetic loci to study gene expression and regulation. The use of chicken DT40 cells to produce modified human chromosomes by homologous recombination is described in, for example, Dieken et al., “Efficient modification of human chromosomal alleles using recombination-proficient chicken/human microcell hybrids,” Nature Genet. 12(2):174-82 (1996).

### Antibody Diversification in Chickens

As noted above, the mechanism of antibody diversification in chickens is strikingly different from that of mammals, such as humans. In chickens, the bursa of Fabricius is critical for the normal development of B lymphocytes. The bursa is productively colonized during embryonic life by a limited number of B cell precursors that have undergone the immunoglobulin gene rearrangements required for expression of cell surface immunoglobulin Immunoglobulin gene rearrangement occurs in the absence of terminal deoxynucleotidyl transferase, has only a single copy of each gene segment to rearrange (with the exception of multiple D gene segments) and therefore generates minimal antibody diversity. In addition, observations that immunoglobulin heavy and light chain variable gene rearrangement occur at the same time and that allelic exclusion of immunoglobulin expression is regulated at the level of variable region gene rearrangement provide a striking contrast to rodent and primate models of immunoglobulin gene assembly. Following productive colonization of the bursa, developing B cells undergo rapid proliferation and the immunoglobulin V region genes that generate the specificity of the B cell surface immunoglobulin receptor undergo diversification through a process called gene conversion. Immunoglobulin diversity in chickens is generated by somatic gene conversion events in which sequences derived from upstream families of pseudogenes replace homologous sequences in functionally rearranged immunoglobulin heavy and light chain variable region genes. This mechanism is distinct from and much more efficient than mechanisms of antibody diversification seen in rodents and primates.

### “Knocking Out” the Endogenous Chicken Light Chain

The procedures resulting in chickens that produce antibodies with chimeric light chains involve “knocking-out” the endogenous chicken light chain gene loci and replacing them with loci encoding the human light chain through homologous recombination. The '681 application and U.S. patent application Ser. No. 11/977,538 teach the functional disruption of endogenous immunoglobulin genes in chicken embryonic stem cells, resulting in the production of chimeric chickens in which endogenous immunoglobulin production has been “knocked-out.” U.S. patent application Ser. Nos. 12/192,020, 11/977,538 and 10/104,486 describe vectors suitable for performing such “knock-outs.” As noted earlier, all of these applications have been incorporated herein by reference. For the sake of concision, their respective disclosures will not be repeated herein, but a few highlights are noted. The '681 application reports that the endogenous avian immunoglobulin gene was knocked-out or rendered functionally disrupted. The application describes how to insert expression cassettes into plasmids to form IgL and IgH targeting constructs, how to release fragments of regions flanking the 5′ and 3′ portions of the chicken IgL by enzymatic digestion, and how to form these flanking regions into plasmids for transfection. The application further describes how to transfect chicken embryonic stem (“ES” or “cES”) cells and select transfected cells. The targeting constructs permit disruption of the endogenous avian immunoglobulin genes in the ES cells by homologous recombination. Vectors have been made that produce small deletions in the IgL locus (targeting the J and C regions) or large deletions (targeting the entire IgL locus).

Introducing Human IgK Variable Chains into the Chicken IgL Locus

### A. Homologous Recombination

Nucleic acid sequences encoding human Ig variable chains can be introduced by any method known in the art. In some preferred embodiments, the nucleic acid sequences are introduced by homologous recombination, in which the nucleic acid sequence one wishes to introduce (the donor sequence) into the target sequence (here the chicken IgL locus) has sequences flanking the sides of the donor sequence that are similar or identical to sequences flanking the target sequence, thereby facilitating exchange of the donor sequence for the portion of the target sequence between the similar or identical flanking sequences. In some preferred methods, the homologous recombination is carried out by use of the well characterized system by which the virus known as “lambda phage” integrates into the genome of E. coli host cells. Lambda phage integrates into the target genome by an integration event at a specific attachment site, att^{λ}. The bacterial attachment site sequence is called attB, while the phage attachment site sequence is called attP. The sequences of these sites are well characterized. Versions of these sequences, modified to increase recombination efficiency, are commercially available from Invitrogen Corporation (Carlsbad, Calif.) as the Gateway® cloning and expression system. The technology provides attB, and attP sequences (as well as attL and attR sequences) for use in cloning genes and sequences into vectors for expression, which Invitrogen states permit ready shuttling of genes or sequences of interest to destination vectors to create expression clones, with the gene or sequence remaining in correct orientation and reading frame. The Gateway® technology includes a number of expression vector and destination vector products to utilize and exploit the homologous recombination possibilities permitted by the att sequences. According to Invitrogen's fact sheet on the Gateway® technology, within each of the Gateway® att recombination sites is a 25-base pair (sometimes abbreviated “bp”) region where the recombination events occur. The Gateway® version of attB (used in expression vectors and expression clones) is 25 bp in length, while that of attP (used in donor vectors) is 200 bp, that of attL (found in entry vectors and entry clones) is 100 bp, and that of attR (used in destination vectors) is 125 bp in length. Invitrogen also sells reaction mixes useful for facilitating homologous recombination reactions using the Gateway® att sequences. See generally, Hartley et al., DNA cloning using in vitro site-specific recombination, Genome Res., 10(11):1788-95 (2000).

B. Replacement of the Chicken IgL V Region with Human IgK V Region Constructs

In preferred embodiments, the endogenous chicken immunoglobulin light chain locus is “knocked-out,” as described above. In the course of knocking-out the endogenous locus, an attP site is inserted into the light chain locus. The knocked-out cells, which can be ES cells and in preferred embodiments are primordial germ cells (“PGCs”), are transfected with human V region constructs, which are designed to integrate into the attP sites previously placed in the light chain locus. For example, an insertion vector carrying an attB sequence and a functional human kappa or lambda light chain sequence would be able to integrate into an attP site previously integrated in the chicken immunoglobulin light chain locus with a portion of the chicken light chain gene deleted.

To create the transgenic chickens of the invention, the insertion vector was uniquely designed based on the immunoglobulin production process in chickens. It contains the attB sequence for integration, an array of human pseudogenes and a functional human light chain sequence. The distinct mechanism of antibody diversification in chickens requires regulatory elements critical to such a mechanism in the immunoglobulin loci to be intact. Since these regulatory elements are not well defined, we chose to modify the loci as little as possible. Our overall strategy involves replacement of the chicken coding sequences with human coding sequences by gene targeting of the endogenous loci, keeping the chicken regulatory regions for optimal expression and regulation in the chicken B cell. Since the loci are large, the replacements were done in sequential steps. The first step was the humanization of the variable regions that resulted in the expression of “chimeric” antibodies with human variable regions and chicken constant regions.

Insertion of the human VK region into the chicken V locus was done using phiC31 integrase to insert a human V insertion vector into an attP site inserted in the chicken IgL gene locus at the time of the deletion of the chicken IgL gene. The human V insertion vector consists of five major parts: a human pseudogene array; chicken IgL regulatory sequences; a human functional VK gene; a chicken IgL constant region; an attB site for insertion; and a β-actin promoter or other chicken promoter active in PGCs (for example, a constitutive promoter other than that of β-actin) for expression of a selection marker. Other versions of the attB site can also be used. Thus, the chicken regulatory elements drive expression of the chimeric human-chicken light chain, and designed human pseudogenes participate in gene conversion. The human IgK locus is considered to be “probably the most complex of all antigen-receptor loci because of the presence of many elements that can be involved in V(D)J recombination.” Langerak and van Dongen, Crit Rev Immunol, 26(1):23-42 (2006). Further, the organization of the human kappa chain gene is more complex than that of the human lambda light chain. Accordingly, the ability to create functional polyclonal antibodies using the coding region for the IgK variable region indicates that the variable region of a human lambda light chain would likewise function in the constructs and methods of the invention.

### Construction of Human Pseudogene Arrays for Gene Conversion in Transgenic Chickens

The invention provides human pseudogene arrays suitable for the production of polyclonal antibodies in transgenic birds, such as transgenic Galliformes and, more particularly, transgenic chickens. During avian B cell maturation, antibody diversity is generated by interchromosomal recombination called gene conversion, or “GC”. The donor sequences in gene conversion come from a series of pseudogenes, which have high homology to portions of the IGVL gene and which are positioned in the genomic sequence near the IGVL gene. The chicken light chain gene locus has a cluster of 25 pseudogenes upstream of the functional V gene segment. See, e.g., Arakawa and Buerstedde, Developmental Dynamics, 229(3):458-464(2004); Reynaud Calif., et al., Cell 48: 379-388 (1987). As summarized by Arakawa and Buerstedde, (1) only the pseudogenes on the same chromosome are used as donors, (2) pseudogenes that are either more homologous, closer or in the opposite orientation to the rearranged V segment are preferred, (3) conversion tracts range from 8 bp to around 200 bp, (4) the 5′ ends of the gene conversion tracts always begin in regions of homology between the pseudogene donor and recipient V segment, whereas the 3′ ends can occur in regions of nonhomology and often encompass nucleotide insertions or deletions, and (5) these results suggest a 5′ to 3′ polarity in the gene conversion mechanism. It should be noted that pseudogenes do not necessarily code for a protein. Their function is as sequence donors to the rearranged and protein coding V-J. If they do contribute sequence to the rearranged V-J then they become part of a coding sequence.

The present invention provides artificial pseudogenes derived from human immunoglobulin gene sequences. A pseudogene of this invention is a nucleotide sequence, typically 20 to about 1000 nucleotides in length, having homology, e.g., 40% to 100% homology, to a genomic nucleotide sequence of some or all of a framework region or CDR of an antibody, e.g., a human antibody, but itself not encoding a full framework region or CDR, which sequence is capable, when properly positioned in the genomic locus normally encoding an avian immunoglobulin chain, of participating in the interchromosomal recombination process of gene conversion. Homology can be at least 50%, at least 60%, at least 70%, at least 80% or at least 90%.

Since the endogenous chicken gene contains 25 pseudogenes, it is clear that gene diversity can be generated using at least that number, and that the cellular machinery that performs gene conversion can work with that many pseudogenes. It is believed that a cluster of as few as about 5 pseudogenes (with “about” meaning one pseudogene more or less) up to about 95 (with “about” meaning 10 pseudogenes more or less) can be used in the inventive methods. For the present work, however, for ease of synthesis and introduction into the chicken variable chain gene locus, it was decided to design an exemplar construct of 10 human pseudogenes. As noted, however, in some embodiments, the construct could be of 7, 8, or 9 pseudogenes, while in others, it could be of 11, 12, 13, 14, 15, 16, 17, 18, 19, 20, 21, 22, 23, 24 or 25 pseudogenes, in some embodiments, 26-85 pseudogenes, in some embodiments, 26-65 pseudogenes and in some embodiments, 26-50 pseudogenes.

The pseudogenes used in this invention can be random sequences having sufficient homology to undergo recombination in an avian B cell. Also, the pseudogenes can be derived from sequences of variable chains of expressed antibodies. For example, these can be portions of variable kappa chains, e.g., human variable kappa chains. For example, the sequences can derive from the immunoglobulin variable kappa germline genes VK2 to VK30, e.g., VK1-33, VK1-39, VK3-11, VK3-15, or VK3-20. In some embodiments, the human sequence is a human lambda light chain variable gene sequence or a sequence derived from a human lambda light chain variable gene sequence. For example, the human lambda light chain variable gene sequence is selected from VL1-44 and VL1-51.

The exemplar construct is comprised of 10 pseudogenes based on human immunoglobulin light chain VK3-20. One hundred expressed sequence tag (EST) sequences were retrieved from the National Center for Biotechnology Information (NCBI) database by BLAST searching with the germline human VK3-20 gene. For each complementarity determining region (CDR), 10 different sequences from 10 different ESTs were selected. In most cases, for each pseudogene, the CDR1, CDR2 and CDR3 were taken from different ESTs. Thus, the methods of selection permitted up to 30 different ESTs to be used in selecting the CDR sequences. For the framework regions, the germline VK3-20 sequences were used for each pseudogene.

In the exemplar construct framework regions were matched and CDR sequences were derived from ESTs. However, CDR sequences provide antibody diversity and can be derived from other sequences or even random sequences. Other VK or variable lambda framework sequence could be used to construct the pseudogene array, but to achieve the homology that facilitates gene conversion, the framework sequence chosen for the functional V gene and that of the pseudogenes should be matched. For example, in the exemplar constructs used in the studies reported in the Examples, the functional V gene and the pseudogenes were both derived from framework sequence VK3-20. If, instead, framework sequence VK3-15 is used to construct the expression cassettes, both the functional V gene and the pseudogenes should be derived from that framework sequence.

Since chicken pseudogenes are interspersed with unique sequence “spacers,” the human pseudogene array was designed to contain spacer sequences between each pair. In the exemplar constructs, 50 bp of spacer sequence was used (although the discussion is of a single strand, for ease of reference, lengths of nucleic acid bases in these constructs will be referred to herein as “bp”), derived from the spacers between endogenous chicken IgL pseudogenes. Different spacers, of different lengths, for example, from 5-500 bp in length, can be used. In some embodiments, the spacers are 10-200 bp. In some embodiments, the spacers are 20-150 bp. In some embodiments, the spacers are 30-125 bp. In some embodiments, the spacers are 40-100 bp. In some embodiments, the spacers are 25-75 bp. All ten pseudogenes were synthesized as individual genes and assembled into an array by recombinant DNA techniques.

To assemble the pseudogenes array to maximize the likelihood they would contribute to gene conversion, all the pseudogene clones were obtained only in direct repeat orientation, never in opposite orientation. Each pseudogene had unique sets of restriction sites on the ends for cloning into an array, and the last pseudogene was synthesized with a loxP site at the 5′ end. After assembly, individual pseudogenes retained the same direct repeat orientation. The human VK functional gene for the constructs was designed based on VK3-20 gene sequence (SEQ ID NO:36) specific for human VK3-20 germline gene, as described above. The VK3-20 gene (SEQ ID NO:36) was synthesized by gene synthesis. To facilitate cloning into the chicken IgL clone, the synthesized sequence included several hundred base pairs of the sequence flanking the chicken V region, to extend convenient naturally occurring restriction sites present in the chicken IgL locus. Using these restriction sites, the human V sequence was cloned into the remainder of the chicken IgL backbone. Thus the transgene replaces the chicken V region coding sequences with the designed human VK coding sequence.

Two versions were made: Sequence 1A (SEQ ID NO:39) contains a human V leader exon directly fused at the DNA level to the human V region exon; in Sequence 1B (SEQ ID NO:40), the two exons are separated by the complete 125 bp chicken intron found between the chicken V leader exon and the chicken V gene. The gene structure of human VK genes and chicken VL genes includes an intron in roughly the same position in the genes.

Preservation of Regulatory Elements within the Chicken IgL Gene

PCR was used to amplify the 5′ region of the locus between the first pseudogene and chicken C region. This region contains the IgL promoter and other regulatory elements. The chicken constant region was also amplified, after which two PCR products were assembled into a 6905 bp chIgL sequence stretching from just downstream of the first pseudogene to just downstream of the chicken IgL constant region. This sequence includes all of the light chain coding elements (V, J, and C regions) as well as all of the introns, but none of the chicken pseudogenes. This sequence was used as the backbone in which the chicken light chain V region was replaced with the human V region. Thus, the chicken IgL promoter and intron sequences drive the expression of the chimeric human VK coding sequence and chicken C region.

Expression of Chimeric IgL mRNA and Protein.

Splicing is a modification of RNA after transcription, in which introns are removed and exons are joined. This is needed for the typical eukaryotic messenger RNA before it can be used to produce a correct protein through translation. Two versions of insertion vector were made as described above. Because of the unique design, it was important to make sure that the splicing of mRNA was as expected and that the mature mRNA carried the correct genetic information for producing a functional chimeric light chain. The studies herein demonstrate that properly spliced transcript was produced by the blood lymphocytes of IgL KI7B chickens (carrying Seq1B version) and IgL KO DT40 cells (carrying either Seq1A or 1B). The spliced transcript of the chimeric IgL product was cloned and sequenced, and its identity and the splicing junctions between the human V leader exon, the human V coding exon, and the chicken C exon were correct. In a separate experiment, chimeric IgL protein with correct molecular weight was detected in IgL KO DT40 cells using Western blotting analysis, demonstrating successful expression of chimeric IgL protein.

Modification of Functional huVK Gene by Gene Conversion in B Cells of Transgenic Chickens.

Gene conversion (GC) is one of the fundamental differences in antibody production between human and chicken. GC contributes significantly to diversification of polyclonal antibodies in chicken B cells. To determine whether gene conversion occurred in B cells of IgL KI7B chickens, RT-PCR was performed on total mRNA from peripheral blood lymphocytes to amplify the human V region coding sequence. The PCR products were then cloned and 184 independent clones were sequenced.

Alignment analysis of the sequences was done with the input, germline sequence that was introduced in the huVK insertion vector, and CDR sequences were compared to the pseudogene CDRs. The sequences from the blood lymphocytes showed that gene conversion had modified the functional human V, using donor sequences from the upstream human pseudogene array. All CDRs showed evidence of gene conversion, and all 10 pseudogenes were used in gene conversion of at least some of the sequences. Point mutations were also observed, in both the framework and CDR sequences. Many sequences showed multiple gene conversion events throughout the V region. Sometimes, a single sequence showed gene conversion by different pseudogenes, confirming independent gene conversion events. Results included in this invention showed that gene conversion is ongoing and cumulative in the B cell population. In addition to the CDRs, there were three base pairs in the framework regions that were present in all the pseudogenes. For purposes of the studies herein, it was considered a gene conversion event if the base pair was observed to be identical to the pseudogene sequence, because although it is a point mutation, it is likely from gene conversion. The gene conversion frequency appears to be lower in CDR3 than in CDR1 or 2. The J sequences present in the human pseudogenes downstream of CDR3 could affect the gene conversion frequency in CDR3. The point mutation frequency appears to be higher in CDR3 than elsewhere in the V region, which could be a result of the decreased gene conversion. Of 184 sequences analyzed, only 3 showed no evidence of any mutations.

### Functionality of the Chimeric IgL.

A functional light chain has to be able to pair with a heavy chain and the hypervariable regions (also called complementarity determining regions, or CDRs) have to form a functional antigen binding site, while the remainder of the VL acts as a scaffold that supports the three-dimensional structure of the antibody. Because the chimeric IgLs described herein are comprised of human variable and chicken constant regions, the question was whether they could pair appropriately with endogenous chicken heavy chains in the transgenic chickens. Using antibody stains against human IgK, chicken IgL, and chicken IgM to detect the chimeric light chain and chicken heavy chain in DT40 cells, only in the chIgL-huV knockin cells was cell surface staining with all three antibodies observed, indicating that the chimeric light chain protein folds properly, binds to the wild-type chicken heavy chain, and traffics to the cell surface as a B cell receptor.

### EXAMPLES

### Example 1

### Normal Birth, Development, and Sexual Maturity of Heterozygous IgL Knockout Chickens

Following artificial insemination of wild type Barred rock hens with semen of IgL knockout (KO-07) roosters, the fertilized eggs were allowed to grow to day 14. At day 14 the embryos were humanely sacrificed. Black feathered embryos were evaluated for genotype by Southern analysis. For genotyping, genomic DNA samples were prepared and digested with SacI restriction enzyme and fractionated on 0.7% agarose gels. Then DNA was transferred to nylon membrane and hybridized with a probe from the chicken IgL locus upstream from the regions. This probe is a 0.5 kb SacI-BstEII fragment this is external to the homology arms. It detects a wild type fragment of approximately 10 kb and a mutant fragment of approximately 4 kb. The probe detected that IgL knockout (IgL KO/+) was transmitted to 5 of the 7 embryos tested, as described in U.S. Patent Application Publication No. US 2010/0138946 (hereinafter, the “'946 patent publication”). In separate experiments, black feathered embryos were incubated until hatching. Combs of hatched black feathered chicks were collected on the first day after birth and genotyping was performed by PCR using the following 2 sets of primers: CLC2F/CLC1R for WT chIgL and ERNI+79F/neo3 for ERNI-neo.

CLC2F:
(SEQ ID NO: 1)
AGACCCTCGGACATCCCTTCACG
CLC1R:
(SEQ ID NO: 2)
AAAACCCCCAAATCACCAAAAATC
ERNI+79F:
(SEQ ID NO: 3)
ACGACAGACTTGAGGGGTTCTC
neo3:
(SEQ ID NO: 4)
GCTCTTCAGCAATATCACGG

(“ERNI” is an abbreviation for the “early response to neural induction” promoter.) The size of the wild type fragment is about 2.1 kb and the mutant is about 800 bp. Results showed that the transgene was detectable in approximately 50% of the offspring, indicating Mendelian inheritance. These IgL KO/+ chickens developed normally and reached sexual maturity at the expected time. There was no sign of immunological diseases or immune incompetency.

### Example 2

### Breeding of IgL Knockout Chickens to Homozygosity

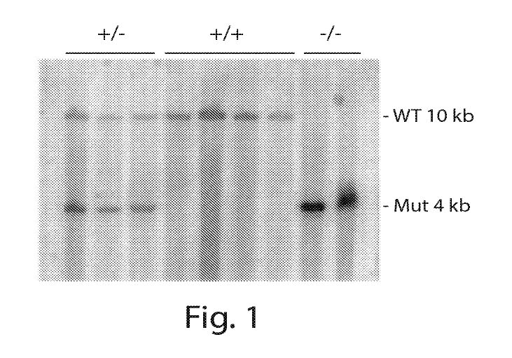

Sexually matured IgL KO/+ roosters were mated to IgL KO/+ hens by artificial insemination. Nine embryos were euthanized at day 3. For genotyping, genomic DNA samples were prepared and Southern analysis was performed using the same 0.5 kb SacI-BstEII fragment as a probe, as described in Example 1. The probe detected only a wild type fragment in 4 (IgL+/+) of the 9 embryos and a mutant fragment in 2 embryos (IgLKO/KO). Both wild type and mutant fragments were detected in 3 embryos (IgLKO/+) (FIG. 1).

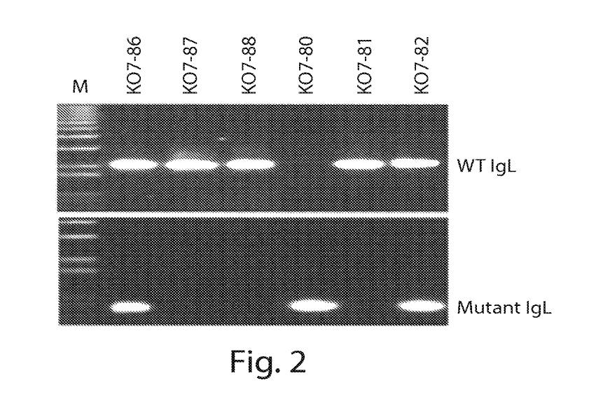

In separate experiments, embryos were incubated until hatched and genomic DNA was prepared from the combs of the chicks on the first day. PCR genotyping was performed using the same 2 sets of primers described in Example 1. In a set of 6 chicks genotyped, three samples produced only wild type IgL fragment of 2.1 kb and 1 produced only ERNI-neo fragment of about 750 bp while 2 produced both wild type and ERNI-neo fragments (FIG. 2). During a period of about 4 months, 89 chicks were genotyped using this PCR and approximately 47% of chicks had 1 mutated allele and 28% had both alleles mutated, indicating Mendelian inheritance (Table 1).

TABLE 1
Offspring from breeding heterozygous chickens carrying
IgL small deletion

| | IgL+/+ | IgLKO/+ | IgLKO/KO |
|---|---|---|---|
| At hatch | 19 | 29 | 15 |
| 2 weeks | 3 | 13 | 10 |
| Total: | 22 (25%) | 42 (47%) | 25 (28%) |

### Example 3

### IgL Mutant (KO/KO) Chickens Lack Peripheral B Cells

It is well established that a functional B cell receptor is necessary for the progression of B cell development. The bursa of Fabricius is productively colonized during embryonic life by a limited number of B cell precursors that have undergone the immunoglobulin gene rearrangements required for expression of cell surface immunoglobulin. Then, developing B cells undergo BCR-dependent rapid proliferation. Because a light chain is necessary to form a functional B cell receptor, the status of B cell development would provide direct evidence as to whether the chicken IgL gene is functionally inactivated in the IgLKO/KO chickens.

To examine this, bursal cells were collected from the Bursa of Fabricius of chicks at hatch. Briefly, single bursal follicles from newly-born chicks were crushed with the plunger of a 1-ml plastic syringe in round-bottomed wells of a microtitration plate, the cells were suspended in 400 ul of 10 mM Tris-HCl, pH 8.0/0.15 M NaCl/10 mM EDTA in an Eppendorf tube. The yield of cells per follicle was between 10⁵ and 3×10⁵, depending on the age of the chicken. Viability of the cells as measured by trypan blue exclusion was around 70-85%. Blood samples were collected from the wing veins of wild type, IgLKO/+ and IgLKO/KO chickens. The blood samples were first mixed with an equal volume PBS and then an equal volume of Histopaque®-1077 (Sigma-Aldrich, St. Louis, Mo.) was layered under the PBS/blood mixture. The tubes were then spun at 1600 rpm for 20 minutes. The middle layer of cells, containing the lymphocytes, was removed and washed with PBS containing 1% FBS. Cells were then stained with PE-conjugated antibody for Bu-1, a commonly used chicken B cell marker, and analyzed by flow cytometry.

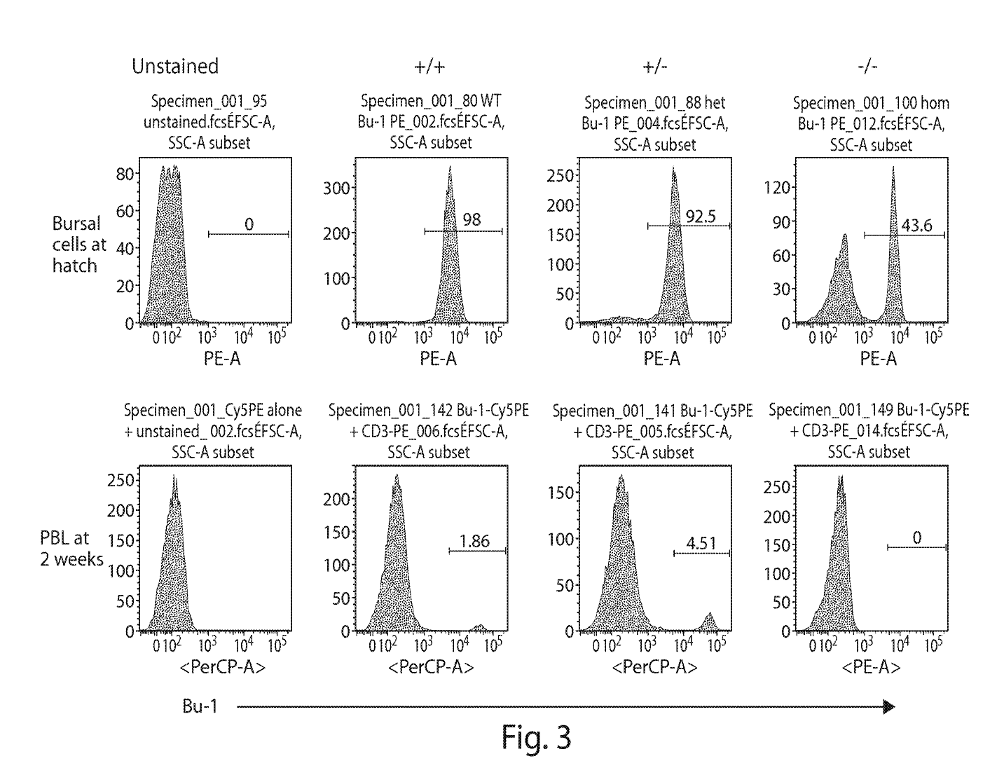

Histograms showed that 98% of wild type bursal cells and 92.5% of IgLKO/+ expressed Bu-1 while only 43.6% of IgLKO/KO bursal cells were Bu-1+ at hatch. FIG. 3 demonstrates, however, that these Bu-1+ cell do not migrate to the peripheral blood and do not produce antibodies. In peripheral blood, no Bu-1+ cells were present in IgLKO/KO chicken comparing to 1.8% and 4.5% in wild type and KO/+ chickens, respectively, at 2 weeks of age (FIG. 3). Thus, the IgLKO/KO chickens are unable to produce Bu-1⁺ B cells because they are missing a required element of the light chain, the J region, and unable to produce a light chain, which is necessary to form a functional B cell receptor. These results indicate that the IgL gene is indeed functionally inactivated by gene targeting and that IgLKO/KO chickens could be a useful vehicle for expressing heterologous antibodies.

### Example 4

### Inactivation of IgL Gene in Chicken DT40 Cells

Knockout animal models provide information on the importance of a disrupted gene in B lymphocyte development. However, an early block in the development or a lethal phenotype prevents the studies of the functional importance of the gene at the later developing system such as the immune system. The chicken B cell line DT40 is widely used to study B lymphocyte development, immunoglobulin gene conversion and antibody production. In the studies reported herein, DT40 cells were used to test, first, whether IgL locus in the genome of these cells could be targeted by the IgL targeting vectors and, second, whether the functionality of the IgL gene could be restored by insertion of a humanized IgL gene.

To target the chicken IgL locus in DT40 cells, a targeting vector (IgL pKOSD) was prepared by a strategy similar to that described in the '946 patent publication to delete the endogenous J and C regions upon targeted integration (FIG. 3a). The vector was the same as previously described, except the selectable marker has been changed. The 5′ homology region on the vector consisted of a 2327 bp fragment in the vicinity of the IgL V region, and the 3′ homology region consisted of a 6346 bp fragment from downstream of the C region. The homology arms were cloned from isogenic DNA obtained from the cell line used in targeting transfections. The targeting construct contained one or more ways to disrupt expression, such as stop codon, nonsense sequences, attP site or combinations thereof. The vector also contained a β-actin-neo cassette consisting of the 804 bp neomycin resistance gene under the transcriptional control of the 1356 bp β-actin promoter for expression in DT40 cells. Transfection was done using a Bio-Rad electroporator (Bio-Rad Laboratories, Hercules, Calif.) according to the user's manual and stable transfectants were screened by Southern blots as described in the '946 patent publication. Five clones were confirmed to have their IgL locus partially deleted.

### Example 5

### Construction of IgL Insertion Vector and Chicken IgL Sequences in IgL Insertion Vector

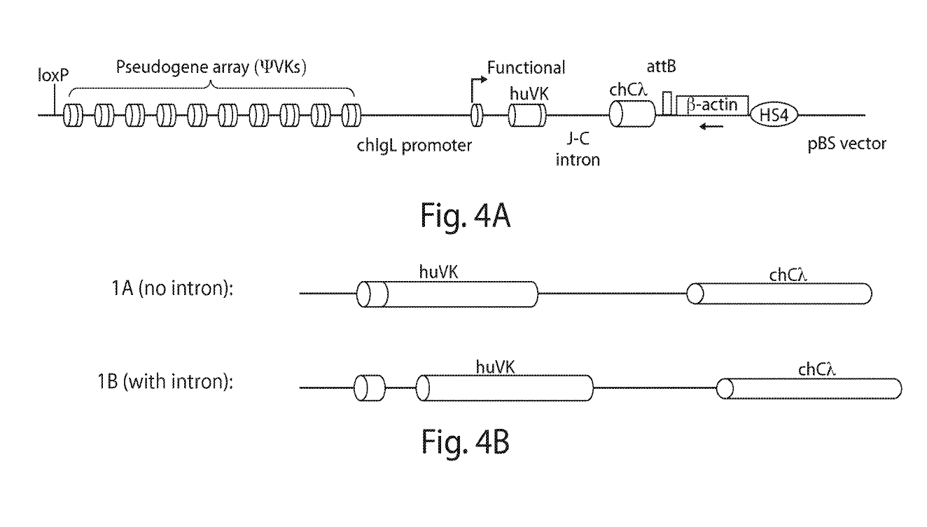

To express a functional human VK gene, an IgLhuVK-attB insertion vector was built. This vector consists of four major parts, including a set of human V pseudogenes, chicken light chain sequences comprising of the IgL promoter, J-C intron, and C region that were deleted upon targeting of the attP site, a functional human VK to replace the chicken light chain V, and the β-actin promoter upstream of an attB site for driving expression of the promoterless puro cassette in the locus (FIG. 4A). This huV insertion vector was used for transfection of PGC lines with either the small deletion or large deletion that were described in the '946 patent publication, since the necessary sequences to reconstitute functionality of IgL gene were the same for both.

The chicken IgL promoter and intron sequences that drive expression of the human VK coding sequence and chicken C region were cloned. First, the chicken IgL region between the first upstream pseudogene and the chicken functional V gene was assembled. This region contains the IgL promoter. PCR was used to amplify the 5′ region of the locus between the first pseudogene and the 5′ SacI site, using genomic DNA from the PGC35 cell line as a template and the following primers:

chIgLpro-F3 tagged with BamHI:
(SEQ ID NO: 5)
aggaTCCTGTAGAGCCTCAGGACTG
R-44:
(SEQ ID NO: 6)
GAACTCTCTATGACCATGGCC

PCR product was cloned and sequenced. Product was then cut with BamHI and SacI and cloned with the PGC35 SacI-SpeI fragment, yielding a 6905 bp chIgL sequence stretching from just downstream of the first pseudogene to the SpeI site just downstream of the chicken IgL constant region. This fragment includes all of the light chain coding elements (V, J, and C regions) as well as all of the introns, but none of the chicken pseudogenes. This fragment was used as the backbone in which the chicken V region was replaced with the human V region.

### Example 6

### Human Functional V Gene Insertion Vector

We used a human monoclonal antibody specific for functional human VL originating from the human VK3-20 germline gene.

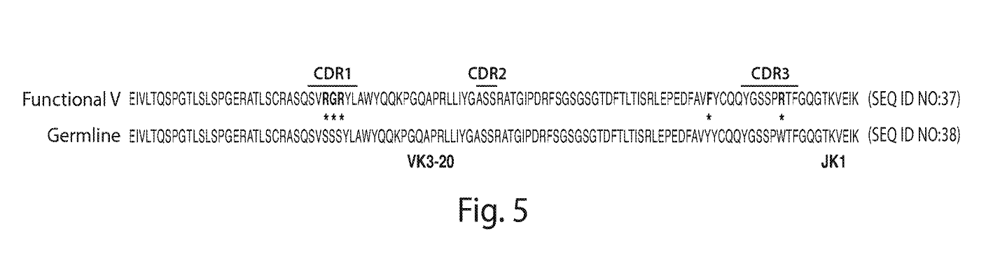

The human VK functional gene was designed based on the VK3-20 germline gene. The VK3-20 was synthesized by BioBasic Canada Inc. (Markham, Ontario) and assembled with chicken regulatory and coding sequences that were previously assembled in Example 5. To facilitate cloning into the chicken IgL clone described in Example 5, the synthesized sequence included several hundred by of the sequence flanking the chicken V region, to extend to convenient naturally occurring restriction sites present in the chicken IgL locus. Using these restriction sites, the human V sequence was cloned into the remainder of the chicken IgL backbone. Thus, the transgene is a perfect replacement of the chicken V region coding sequences with the human VK coding sequence. At the DNA level, this human VK coding sequence has 79 bp in framework region 1 (FWR1) identical to germline VK3-20, 159 bp in FWR2/CDR2/FWR3 identical to germline VK3-20, and 37 bp in J region identical to germline JK1. At the protein level, this human VK coding sequence (SEQ ID NO:37) contains 5 different amino acids compared to germline configuration of human VK (SEQ ID NO:38) (FIG. 5).

Next, we cloned human VK genes into the chIgL sequences obtained in Example 5, using the BmgB1 and SgrA1 sites (naturally occurring sites upstream of chicken V and downstream of chicken J).

### Example 7

### Different Versions of Human Functional V Gene in IgL huVK Insertion Vector

The gene structure of human VK genes and chicken VL genes includes a leader intron sequence in roughly the same position in the genes. The leader intron in the chicken IgL locus is small (125 bp). One version of IgL huVK was made without it, leaving the functional V as one continuous exon. This removes the possibility of splicing errors. Since the intron could possibly contain an important regulatory sequence, we also made a version containing the chicken leader intron (FIG. 4B). The version containing the intron was used first, and there was no indication of splicing errors, which was evident in DT40 experiments, so the version without the intron was not used for transfection of PGC cells.

### Example 8

### Composition of Human Pseudogene Array

Ten pseudogenes were designed and synthesized based on human VK3-20. First, 100

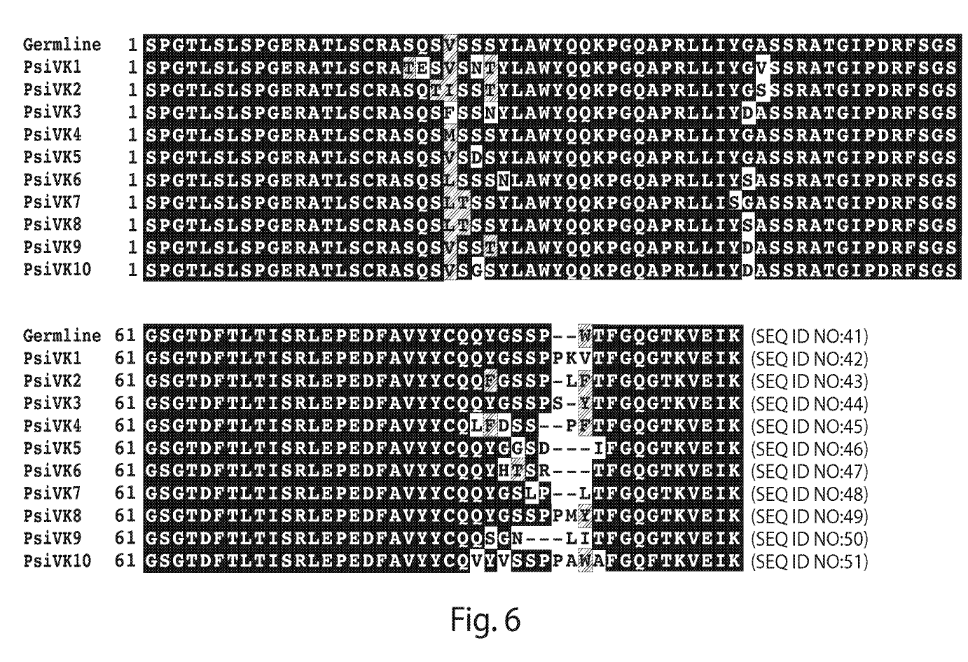

EST sequences from the NCBI database were retrieved by BLAST searching with the germline human VK3-20 gene. Then, an alignment of these 100 human VK3-20 EST sequences was made, and the CDRs were inspected for sequences mutated by human B cells. These mutated sequences were candidates that could be incorporated into the pseudogene pool. For each CDR, 10 different sequences from 10 different ESTs were selected although CDR2 diversity was quite limited and some of the pseudogenes contained the same CDR2 sequence. CDR3 contained the most diversity and a variety of sequences and lengths were selected (FIG. 6). For each pseudogene, the CDR1, CDR2 and CDR3 were usually taken from different ESTs. Thus, about 30 different ESTs were used in selecting the CDR sequences.

The framework regions were all identical to germline human VK3-20. Therefore, for the framework regions, the germline VK3-20 sequences were used for each pseudogene. Since the chicken pseudogenes are interspersed with unique sequence “spacers,” the human pseudogene array was also designed to contain 50 bp of spacer sequence between each pair. These spacer sequences derived from the endogenous chIgL pseudogene array were placed adjacent to each pseudogene (see Example 33 for sequences of the spacers). These spacers served to mimic the endogenous locus, and also provided a unique tag for each pseudogene which was useful as a place where sequencing primers could bind. These 10 pseudogenes were named as pseudogenes 1˜10 (YVK1˜10) and translated to amino acids, which were aligned with the protein sequence of VK3-20. (FIG. 6).

### Example 9

### Unique Assembly of Pseudogene Array

The array was built up from ten individual genes. These individual genes were synthesized and cloned individually in a universal vector pUC57. Each pseudogene had BamHI and BglII sites on the ends and YVK10 had a loxP site at the 5′ end, and NotI and BamHI sites for cloning into an array. Vectors containing YV2, 4, 6 and 8 were digested with BamHI+BglII and 0.3 kb individual pseudogenes were purified; vectors containing YV1, 3, 5 and 7 were digested with BglII to be linearized and dephosphated with CIP. Ligation was performed in groups of two (1+2, 3+4, 5+6, 7+8), and each resulting clone contained two pseudogenes. All the pseudogene clones were only obtained in direct repeat orientation, never in opposite orientation relative to the functional VK. Direct repeat orientation is reportedly more efficient in engaging in gene conversion. The vectors containing 2 pseudogene intermediates (YVK1/2, 3/4) were digested with BglII to be linearized and ligated to individual 2 pseudogene intermediates (5/6, 7/8) that were digested with BamHI. The resulting vectors contain4 pseudogene intermediates (1/2/7/8, 3/4/5/6, etc.). These 4 pseudogene intermediates were assembled to 8 pseudogene intermediates using the same strategy. The final two pseudogenes YVK9 and YVK10, were added separately to the final construct.

### Example 10

### Adaptability of HuVK-attB Insertion Vector

The design of the human V insertion vector was such that the functional V could be swapped using unique restriction sites. The pseudogene pool could remain the same, especially if the framework regions are from VK3-20/VH3-23 or sufficiently similar to enable efficient gene conversion. After knowing that established chIgL-attP PGCs are capable of going germline, the new human V insertion vectors could be easily placed into the locus with integrase. To place a new functional human V into the locus would require a simple cloning step to make the insertion vector, followed by transfection into IgL-attP PGCs and injection of the PGCs. G1 animals would hatch approximately 2.5 quarters from the start of the experiment. Making alternative versions of the human VK region could also be done.

### Example 11

### Molecular Docking Site for Insertion of huVK into the Chicken IgL Locus

We have also used the phiC31 integrase system, which catalyzes site-specific recombination between an attB site and an attP site, to insert foreign DNA into the chicken genome. Recombination between phiC31 attB and attP sites is irreversible, so insertion of a circular construct bearing an attB site into the genome is stable and does not get looped out, even in the continued presence of integrase. It has also been shown that the incoming plasmid must carry an attB site rather than an attP site for efficient integration (Belteki et al., Nat. Biotechnol. 21(3):321-4 (2003); Thyagarajan et al., Mol Cell Biol. 21(12):3926-34 (2001)). Therefore, an attB site was placed directly downstream of the chicken β-actin promoter. A loxP site was also included for the downstream step of removal of the selectable markers. The huVK insertion vector was designed so that, upon insertion into the attP site in the chicken IgL locus, the β-actin promoter would be placed directly upstream of the puromycin gene that previously had no promoter. The puromycin gene would then be expressed and the transfected cells would become puromycin-resistant.

### Example 12

### Schematic Diagram of IgL huVK Insertion into Small Deletion IgL Knockout in DT40 Cells (DT40-attP)

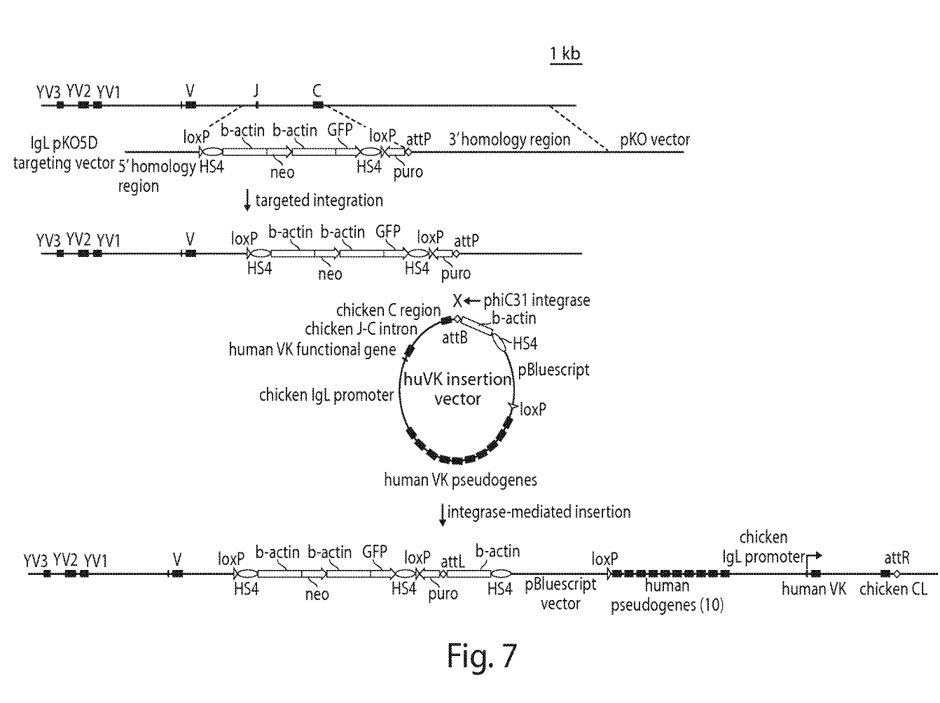

IgLKO DT40 cells were generated previously by transfection of an IgL targeting vector (IgL pKOSD) designed to create a small deletion in IgL locus of DT 40 genome. An attP is also in place for insertion of human VK sequence. The vector was designed so that co-transfection of IgL huVK insertion vector along with phiC31 integrase plasmid would result in insertion of huVK sequence in IgL locus of DT40 cells and activate the expression of previously inserted promoterless puromycin cassette for selection of puromycin-resistant PGC clones that containing the human VK insert (FIG. 7).

### Example 13

### Schematic Diagram of huVK Insertion into Small Deletion IgL Knockout in PGC Cells (IgLKO-07)

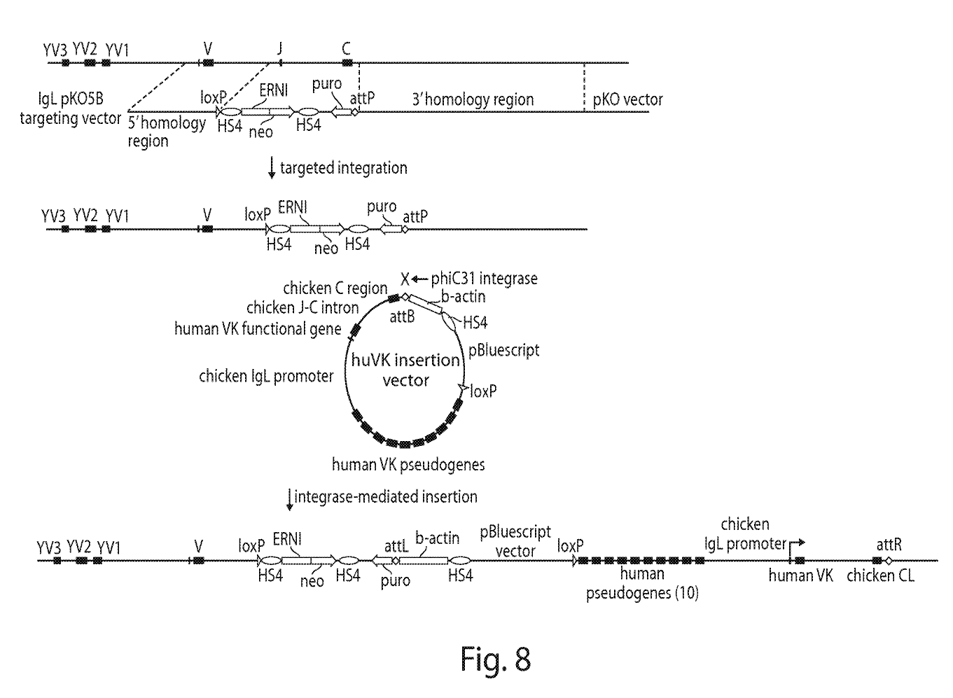

IgLKO-07 PGC cells have a small deletion, missing only the J variable gene segment, in their IgL locus which was created by homologous recombination when an IgL targeting vector (IgL pKOSB) was transfected into the cells. Also inserted was a molecular docking sequence, attP. In the presence of phiC31 integrase, an incoming vector carrying an attB site can irreversibly insert a foreign sequence into attP site. The vector was designed so that co-transfection of the IgL huVK insertion vector along with phiC31 integrase plasmid would result in insertion of huVK sequence into chicken IgL locus. This also positioned the β-actin promoter in place to drive the expression of previously inserted promoterless puromycin cassette for selection of puromycin-resistant PGC clones that would contain human VK insertion (FIG. 8).

### Example 14

### Schematic Diagram of huVK Insertion into Large Deletion IgL Knockout in PGC Cells (IgLKO-12 & KO-13)

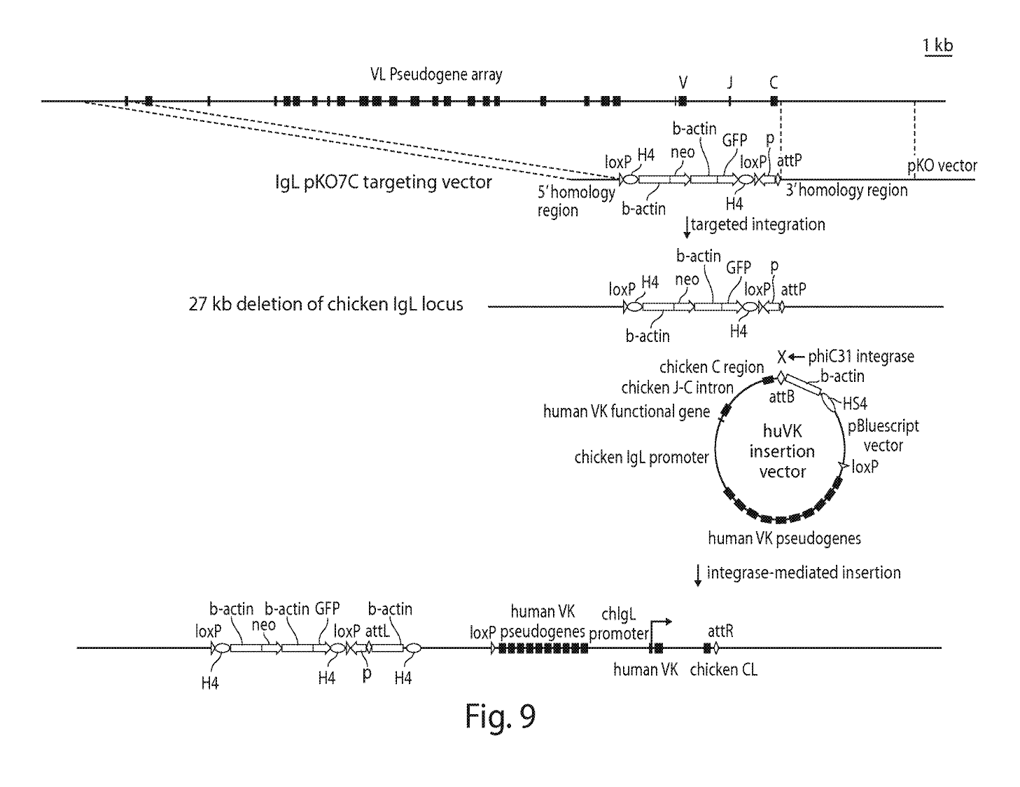

Similarly, IgLKO-12 and KO-13 PGC cells were generated when an IgL targeting vector (IgL pKO7C) was transfected into the cells. Because the 5′ arm of the pKO7C is located upstream of chicken VL pseudogene array, recombination deleted chicken endogenous pseudogenes, and V, J, and C regions. An attP is also present in targeted IgL locus. The vector was designed so that co-transfection of the huVK insertion vector along with phiC31 integrase plasmid resulted in insertion of huVK sequence in chicken IgL locus. This also positioned the β-actin promoter in place to drive the expression of previously inserted promoterless puromycin cassette for selection of puromycin-resistant PGC clones that contained the human VK insertion (FIG. 9).

### Example 15

### Insertion of Human VK into IgLKO DT40

For transfection, 5×10⁶ DT40 cells were resuspended in 0.8 ml PBS at room temperature. 5 μg linearized phiC31 integrase plasmid DNA and 10 μg circular IgL huVK insertion vector DNA were added into cell suspension. The cell suspension was then transferred to a 0.4 cm gap cuvette and electroporation was done at 550V, 25 μF, exponential decay using a Bio-Rad electroporator. The cuvette was incubated for 10 minutes at room temp before diluting the cells into 10 ml DT40 medium and plating in one 96-well plate, 100 μl per well. After 24 hours, selection was applied by adding puromycin to the medium to a final concentration of 0.5 μg/ml. Colonies were visible by 5-6 days of selection, and they were picked after 6-8 days and expanded for PCR genotyping.

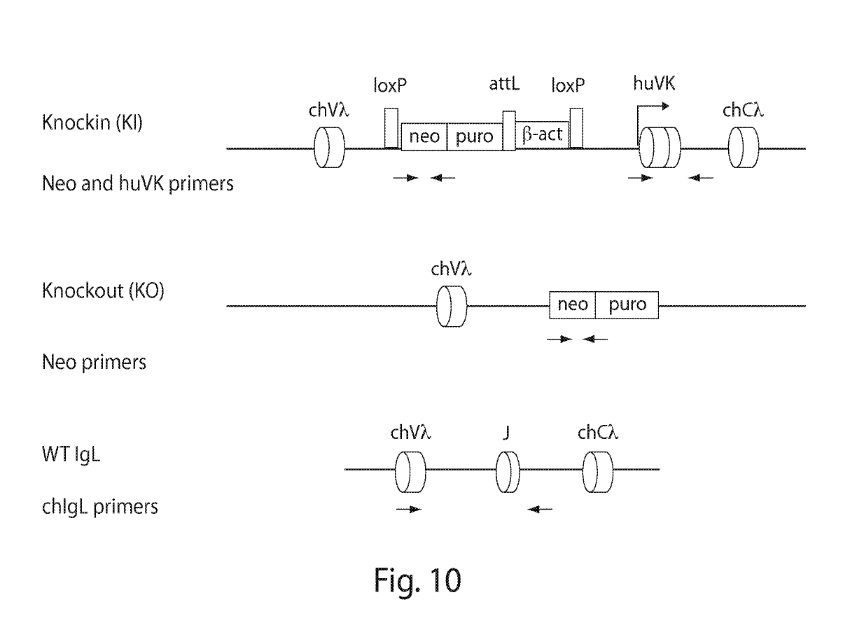

The strategy for PCR genotyping of the insertion of IgL huVK into IgLKO DT40 cells is depicted in FIG. 10. Basically, 3 sets of primers were designed to amplify wild type IgL allele, the knockout cassette, and the huVK insertion, respectively. Primers for the wild type IgL allele were CLC2F and CLC1R; primers for ERNI-neo cassette were ERNI+79 and neo3; and primers for huVK insertion were huVK3-20sig-F and CLC1R. The sequences of these primers are as follows:

CLC2F:
(SEQ ID NO: 7)
AGACCCTCGGACATCCCTTCACG
CLC1R:
(SEQ ID NO: 8)
AAAACCCCCAAATCACCAAAAATC
ERNI+79F:
(SEQ ID NO: 9)
ACGACAGACTTGAGGGGTTCTC
neo3:
(SEQ ID NO: 10)
GCTCTTCAGCAATATCACGG
huVK3-20Sig-F:
(SEQ ID NO: 11)
GCTTCTCTTCCTCCTGCTACTCTG
CLC1R:
(SEQ ID NO: 12)
AAAACCCCCAAATCACCAAAAATC

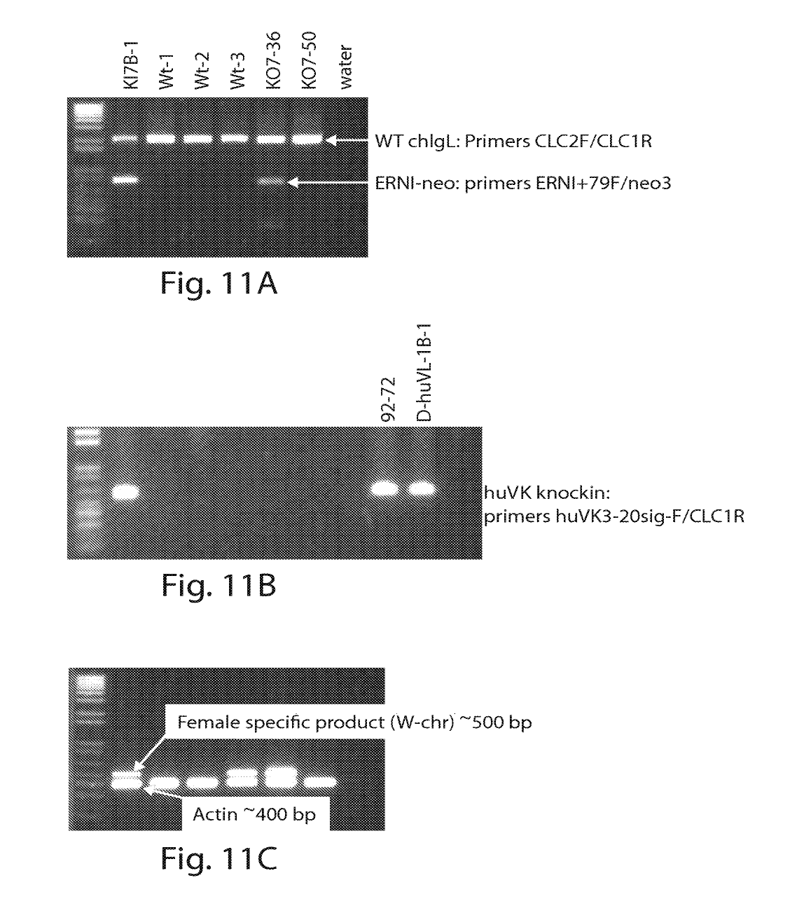

The wild type fragment should be about 2.1 kb; the mutant fragment for ERNI-neo cassette should be 800 bp; and the fragment for huVK insertion should be about 600 bp. The expected PCR results for each of the 4 genotypes are shown in Table 2. Results showed that 3 of 10 clones tested were positive for insertion (FIG. 11).

TABLE 2
Expected PCR results for each of the 4 genotypes

| PCR | WT | KI | KO | KI/KO |
|---|---|---|---|---|
| Human VK (KI) | neg | pos | Neg | pos |
| WT IgL | pos | pos | Pos | neg |
| Neo (KO and KI) | neg | pos | Pos | pos |

### Example 16

### The Outcomes of Human VK Insertion into IgLKO DT40 Cell

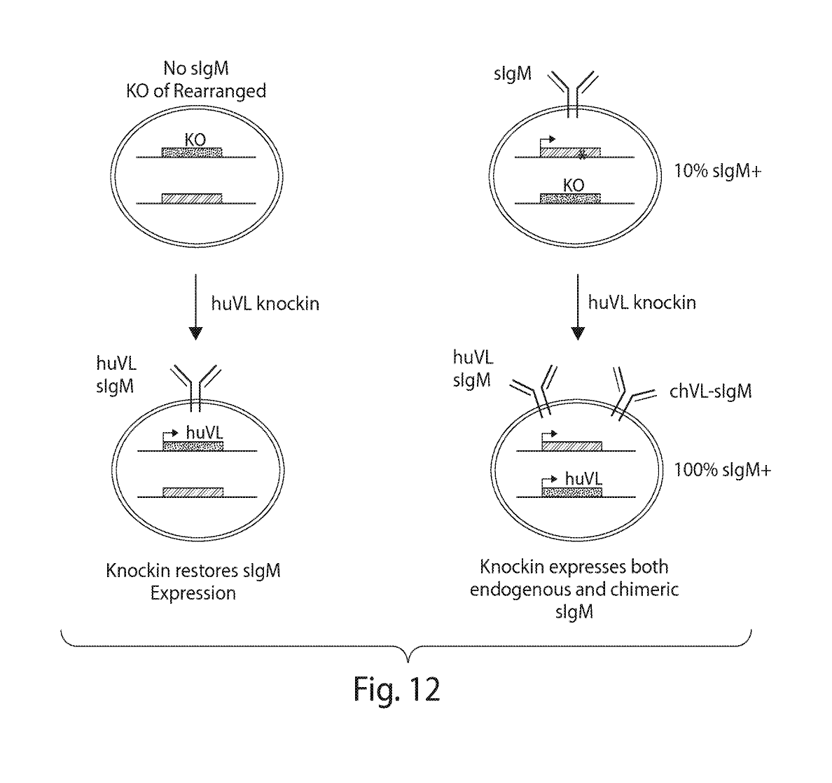

Allelic exclusion is a process by which one allele of a gene is expressed while the other allele is silenced. Allelic exclusion has been observed most often in genes for cell surface receptors and has been extensively studied in immune cells such as B lymphocytes. In B lymphocytes, successful heavy chain gene rearrangement of the genetic material from one chromosome results in the shutting down of rearrangement of genetic material from the second chromosome. If no successful rearrangement occurs, rearrangement of genetic material on the second chromosome takes place. Allelic exclusion is thought to be regulated at the level of gene rearrangement in chicken B cells, whereas it is regulated at the level of protein production in mice and humans. The DT40 cell line has one light chain allele in that has rearranged and one light chain allele in germline configuration. When the rearranged IgL locus in DT40 cells is partially deleted by a recombination with a targeting vector followed by insertion of IgL huVK, insertion of huVK would restore the expression of sIgM; if the non-rearranged IgL locus is deleted, insertion of huVK would lead to expression of chimeric sIgM in DT40 cells that are expressing chicken endogenous sIgM (FIG. 12). Understanding of these possible scenarios helps interpret the IgL expression results when insertion of huVK occurs in IgL KO DT40 cells.

### Example 17

### RT-PCR for Chimeric IgL Expression in DT40 Knockins

Poly A+ mRNA was extracted from DT40 IgL, knockin cells (D-huVL1A-6 and D-huVL1B-1) according to Oligotex Direct mRNA Protocol (Qiagen Inc., Valencia, Calif.) and first strand cDNA synthesis was done using the ThermoScript™ RT-PCR system (Life Technologies, Grand Island, N.Y.). Briefly, 1.0 μl of Oligo(dT)20 (50 ng/μl), 9 μl of poly A+ mRNA from the above procedure, and 2 μl of 10 mM dNTP were added in a 0.2 ml tube and incubated at 65° C. for 5 min, then placed on ice, 8 μl of cDNA synthesis buffer was added to the reaction tube, and the tube was heated at 50° C. for 1 hour on a preheated thermal cycler. After being incubated at 85° C. for 5 min, 1 μl of RNase H was added and the mix was incubated at 37° C. for 20 min. 2 μl of the resulting cDNA was used for PCR amplification with Platinum® Taq DNA polymerase (Life Technologies). The two primers used for amplification of chimeric IgL were huVK3-20 Sig-F and chCL-R and the two primers used for amplification of actin (as controls) were actin RT-F and actin RT-R. The upstream primer (huVK3-20Sig-F) hybridizes to the human V region leader exon, and the downstream primer hybridizes to the chicken constant region (chCL-R). As a control, primers that hybridize to the chicken actin gene (actin-RT1 and actin-RT2) were also used.

Primer sequences for RT-PCR in DT40 huV knockin cells:

huVK3-20Sig-F
(SEQ ID NO: 13)
GCTTCTCTTCCTCCTGCTACTCTG
chCL-R
(SEQ ID NO: 14)
TTCGTTCAGCTCCTCCTTTGACG
huJKrev
(SEQ ID NO: 15)
GTTTGATTTCCACCTTGGTCCC
chIgL intron-R
(SEQ ID NO: 16)
AGAAAGACCGAGACGAGGTCAGC
actin-RT1
(SEQ ID NO: 17)
AACACCCCAGCCATGTATGTA
actin-RT2
(SEQ ID NO: 18)
TTTCATTGTGCTAGGTGCCA

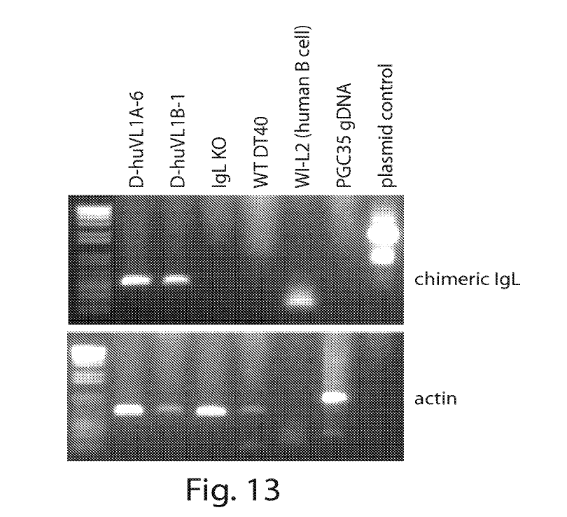

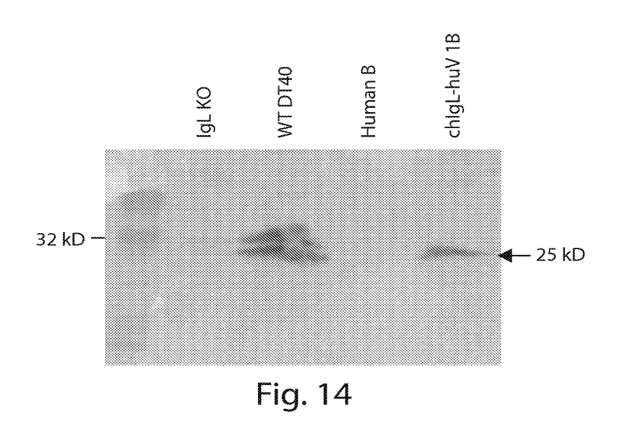

After initial denaturation at 94° C. for 2 min, 35 cycles of PCR were performed by incubating the reaction mixture in the following conditions: 94° C. for 45 sec, 62° C. for 45 sec and 72° C. for 1 min. The PCR products were run on 1% agarose gel. The correct size product was obtained for both sets of reactions (FIG. 13), and the chimeric IgL product was sequenced to verify its correct identity and to verify that the splice junctions between the human V leader exon, the human V coding exon, and the chicken C exon were correct. These results showed that the properly spliced transcript was produced by DT40 cells.

### Example 18

### Detection of Chimeric IgL Protein Expression in IgL huVK DT40 Cells

Western blot analysis of the chimeric IgL expressed in DT40 was performed. Cellular proteins were extracted from cell pellets using lysis buffer (1% NP-40 0.4% deoxycholate, 66 mM EDTA, 10 mM Tris pH7.4). Proteins were separated on denaturing SDS/acrylamide gels and transferred to nitrocellulose membrane. The blots were probed with antibodies against chicken IgY (both heavy and light chain-specific) which contained antibodies specific for the chicken light chain constant region. A single band was observed at about 23 kD, the expected size for the chimeric light chain, and the same size as a band in the wild type control DT40 that was also run on the Western (FIG. 14).

### Example 19

### Detection of Chimeric IgL Protein on the Surface of IgL huVK DT40 Cells

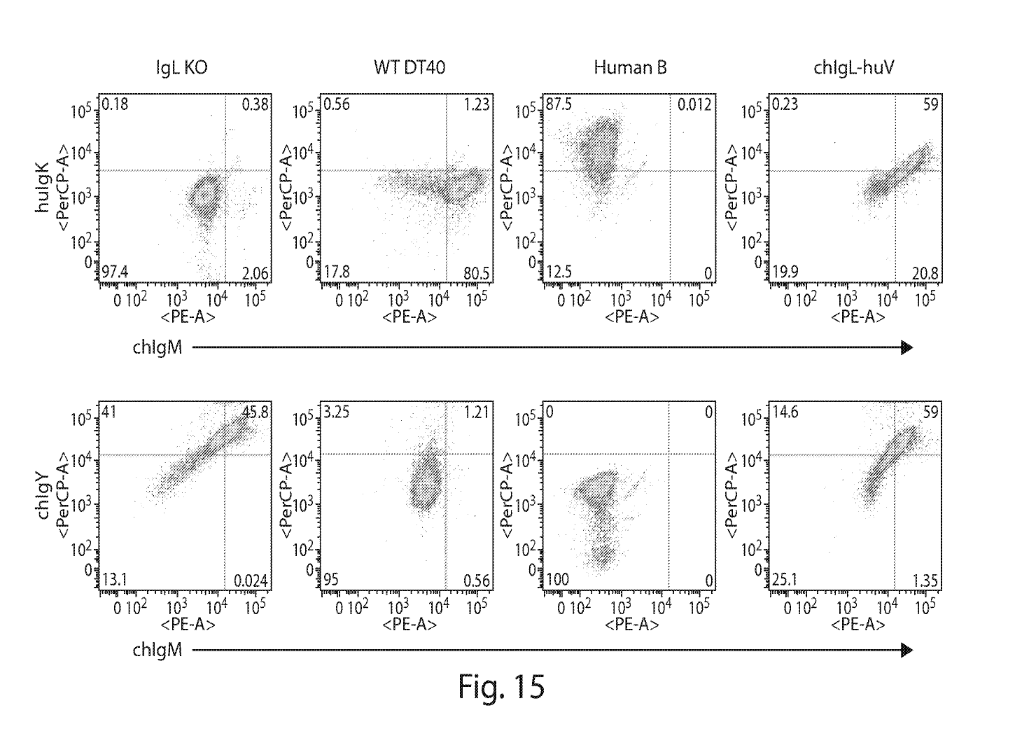

Cultured IgL huVK DT40 cells were collected and antibody stains against human IgK, chicken IgL, and chicken IgM were used to detect the chimeric light chain and chicken heavy chain in DT40. The antibody against human IgK is a goat polyclonal originally against the whole human IgK light chain, therefore some of the individual antibodies in the polyclonal mixture bind to epitopes in the variable region. Similarly, the chicken IgL antibody is a goat polyclonal against chicken IgY (heavy and light chains) but contains some antibodies that bind to the chicken light chain constant region. In control light chain knockout cells, the light chain is absent and thus the heavy chain is retained inside the cell and no cell surface staining with any of the antibodies is observed. In wild type chicken cells, staining is observed with the anti-chicken IgL and IgM antibodies, but not human IgK. In human B cells, staining is only observed with anti-human IgK antibodies. Only in the chIgL-huV knockin cells is cell surface staining with all three antibodies observed (FIG. 15), indicating that the chimeric light chain protein folds properly, binds to the wild type chicken heavy chain, and traffics to the cell surface as a B cell receptor.

### Example 20

### Transfection and Insertion of Human VK into PGCs

To test whether the human VK insertion vector is able to integrate into the attP site within the KO cassette of chIgL knockout vector and whether human VK is functional, IgLKO PGC cells were transfected with the huVK insertion vector and selected for puromycin-resistant clones. IgLKO-07 PGC cells were grown as described (van de Lavoir et al., Nature 441:766-9 (2006)). The Amaxa® Nucleofector® device (Lonza Walkersville Inc., Walkersville, Md.) was used for transfections. 5×10⁶ KO-07PGCs were resuspended in either 100 or 400 ml Amaxa® V buffer. Five mg of circular attB-containing huVL insertion construct was combined with 3 mg of circular integrase DNA. After transfection the cells were grown for several days before puromycin (0.5 mg/ml) was added.

### Example 21

### Screening for Insertion of chIgL-huVK into IgLKO PGCs

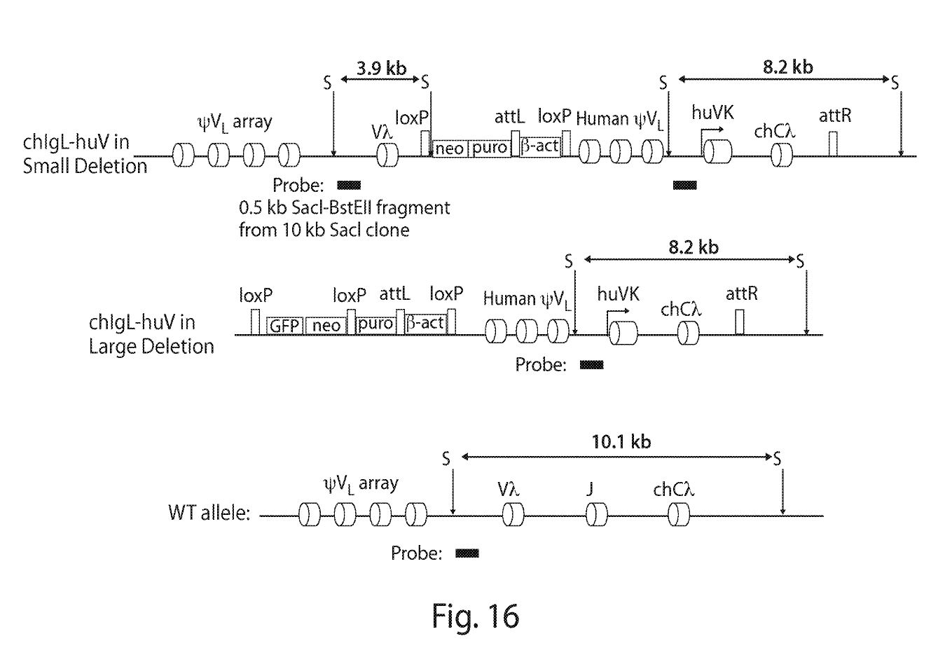

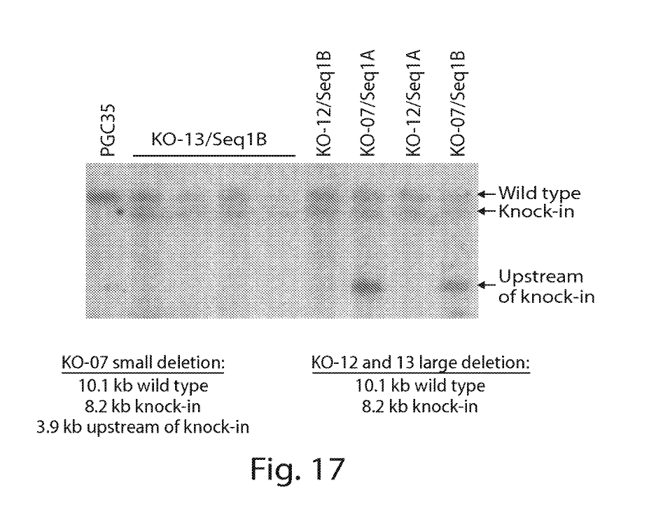

Clones were analyzed by Southern blot for correct insertion (FIG. 16). Briefly, genomic DNA (2 mg for SacI digest; 10 mg for BstEIIdigest) was digested, fractionated on 0.7% agarose gels, transferred by capillary transfer in 10×SSC to nylon membrane and hybridized with ³²P-labeled IgL fragments in Rapid-hyb buffer (Amersham, Piscataway, N.J.). Probe A was a 500 bp SacI-BstEII fragment from the 50 IgL region and Probe B was a 766 by SfiI fragment from the 30 IgL region. After washing, the blot was exposed to film overnight at −80° C. Out of several PGC lines generated from transfection of the huVK-attB insertion vector into large and small IgL deletion PGCs, eight of the clones were verified by Southern analysis (FIG. 17). These clones were named IgLKIB7 cell lines.

### Example 22

### Injection of KI-7B Cell Line

This task was for the establishment of birds carrying the IgL-huVK. Five of eight of the clones verified by Southern blotting were injected into the bloodstream of Stage 14-16 embryos. The embryos were grown, chicks hatched, and the G0 potential germline chimeric males were grown to sexual maturity. The G0 males were test mated to wild type Barred Rock hens to pass the genetic modification on to the next generation and produce fully transgenic chickens carrying the chimeric light chain construct in every nucleated cell of the body. A summary of the chIgL-huV injections is shown in Table 3 above. Fertile eggs from White Leghorn were incubated and the embryos retrieved at stage 13-15 (H&H). One μl containing 3000 PGCs was injected using a 37 μm diameter needle into the anterior portion of the sinus terminals and the injected embryos were transferred to a surrogate shell for incubation until hatch. After hatching, 27 G0 roosters were obtained and will be raised to sexual maturity and bred with Barred Rock hens to obtain roosters and hens that are fully transgenic for the chIgL-huV insert. The appearance of black chicks is indicative of germline transmission of the injected PGCs. The presence of the transgene will be confirmed in black chicks by Southern analysis of comb tissue.

TABLE 3
Summary of huVK knockin cell lines into large and small deletions. Eight
of the clones were verified by Southern and 5 of these were injected.
27 male G0 chimeras were hatched and are being raised to sexual
maturity. n/d = not done.

| | Total chIgL-huV | chIgL-huV | # G0 males |
|---|---|---|---|
| Parental cell line | clones | clones injected | being reared |
| KO-07 small deletion | 47 | 2 | 20 |
| KO-12 large deletion | 25 | 1 | 2 |
| KO-13 large deletion | 50 | 1 | 0 |
| KO-15 large deletion | 17 | 1 | 5 |
| KO-16 large deletion | 5 | n/d | n/d |
| KO-17 large deletion | 1 | n/d | n/d |
| KO-18 large deletion | 17 | n/d | n/d |

### Example 23

### Germline Transmission of IgLKI7B PGCs

Following artificial insemination of semen of 27 G0 chimera roosters with wild type Barred rock hens, chicks were hatched. A total of 1,663 chicks hatched and, among them, there was one black feathered female chick with normal development. Because black offspring could be derived only from PGCs, this chick was tested for the presence of the transgene. PCR genotyping confirmed that it carried the chimeric IgL. The rate of germline transmission was low in this case but black feather-screening made the process easy.

### Example 24

### Breeding and Genotyping of G1 Heterozygous IgL-huVK KI Chickens

This IgLKI chicken was grown to sexual maturity and bred to Barred Rock roosters to generate G1 chickens carrying the transgene. For genotyping, genomic DNA samples were prepared from combs of newly-born chicks and PCR was performed, as described in Example 15. The transgene is inherited by approximately 50% of the chIgL-huVK offspring.

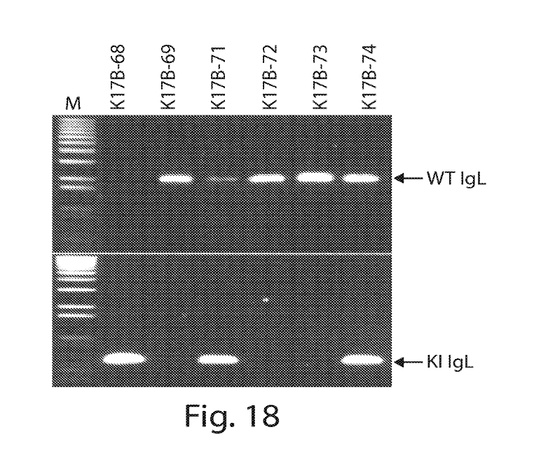

For ease of maintaining the transgenic line, sexually matured IgL KI/+ roosters were mated to IgL KI/+ hens by artificial insemination and their offspring were genotyped at hatch by PCR. Selective set of PCR results are shown in FIG. 18. Table 4 shows a summary of genotyping results of a total of 49 offspring during a period of approximately 3 months. Mating between KI/+ and KI/+ produced about 28.6% of wild type, 44.9% of KI/+, and 26.5% of KI/KI. These results indicated Mendelian inheritance of the chIgL-huVK transgene.

TABLE 4
Offspring of KI7B heterozygous breeding

| | WT | KI/+ | KI/KI |
|---|---|---|---|
| Number at hatch | 14 | 22 | 14 |
| Percentage | 28.6% | 44.9% | 26.5% |

### Example 25

### B Cell Development in chIgL-huVK Chickens

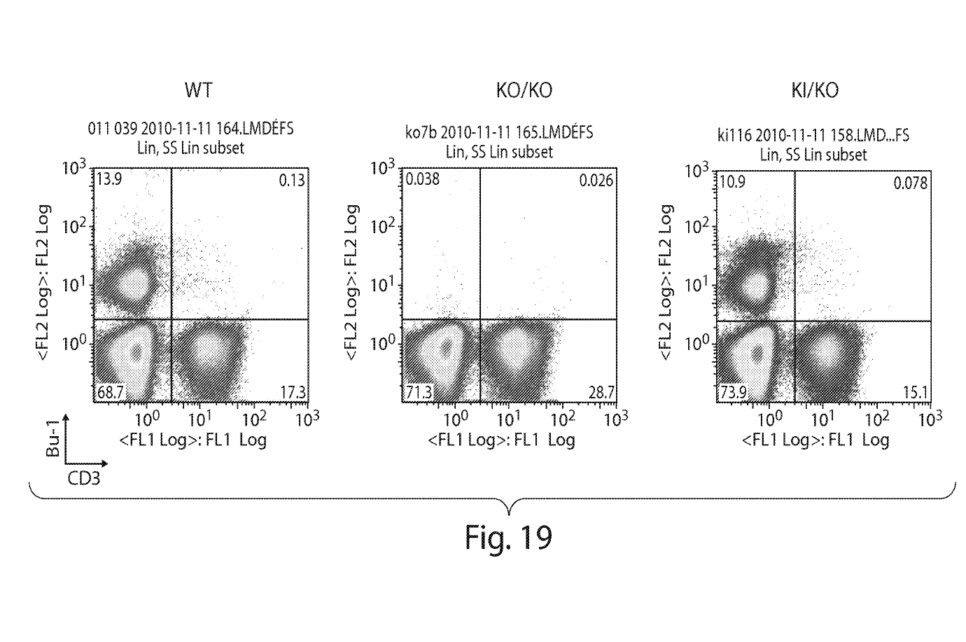

To assess B cell production in KO/KO and KO/KI chickens, lymphocytes isolated from peripheral blood were stained with anti-chicken Bu-1 and anti-chicken CD3 antibodies. Bu-1 is commonly used to identify B cells in chickens, while CD3 is a T cell marker. Lymphocytes from the peripheral blood of wild type chickens were also analyzed as a control. All chickens of the various genotypes produced CD3⁺ T cells, while only the wild type and KO/KI chickens produced Bu-1⁺ B cells (FIG. 19). It is well established that a functional B cell receptor is necessary for the progression of B cell development. As noted above, KO/KO chickens are missing a required element of the light chain, the J region and C region, and therefore are unable to produce a light chain. Insertion of huVK into previously partially deleted IgL locus in KO/KO chickens restored the expression of light chain. Therefore, a functional B cell receptor was formed supporting the development of B cells. The fact that Bu-1⁺ B cells are produced in KI/KO chicken demonstrates that the addition of the human K light chain variable region is able to rescue B cell development in KO/KO chickens.

### Example 26

### Chimeric IgL Expression in huVK Knockin Chickens by RT-PCR

Poly A+mRNA was extracted from lymphocytes of KIM heterozygous chickens according to Oligotex Direct mRNA Protocol (Qiagen) and first strand cDNA synthesis was done using the ThermoScript® RT-PCR system, similar to that described in Example 17. Briefly, 1.0 μl of Oligo(dT)20 (50 ng/μl), 9 μl of poly A+mRNA from the above procedure, and 2 μl of 10 mM dNTP were added in a 0.2 ml tube and incubated at 65° C. for 5 min, then placed on ice. Eight μl of cDNA synthesis buffer was added to the reaction tube and the tube was heated at 50° C. for 1 hour on a preheated thermal cycler. After incubation at 85° C. for 5 min, 1 μl of RNase H was added and incubated at 37° C. for 20 min Two μl of the resulting cDNA was used for PCR amplification with Platinum® Taq DNA polymerase. Two primers were used for amplification of chimeric IgL: huVK3-20 Sig-F and chCL-R and two primers were used for amplification of actin (as controls): actin RT-F and actin RT-R. Primer sequences for RT-PCR in chicken B cells:

huVK3-20Sig-F
(SEQ ID NO: 19)
GCTTCTCTTCCTCCTGCTACTCTG
chCL-R
(SEQ ID NO: 20)
TTCGTTCAGCTCCTCCTTTGACG
actin-RT1
(SEQ ID NO: 21)
AACACCCCAGCCATGTATGTA
actin-RT2
(SEQ ID NO: 22)
TTTCATTGTGCTAGGTGCCA

After initial denaturation at 94° C. for 2 min, 35 cycles of PCR were performed by incubating the reaction mixture in the following conditions: 94° C. for 45 sec, 62° C. for 45 sec and 72° C. for 1 min. The PCR products were run on 1% agarose gel.

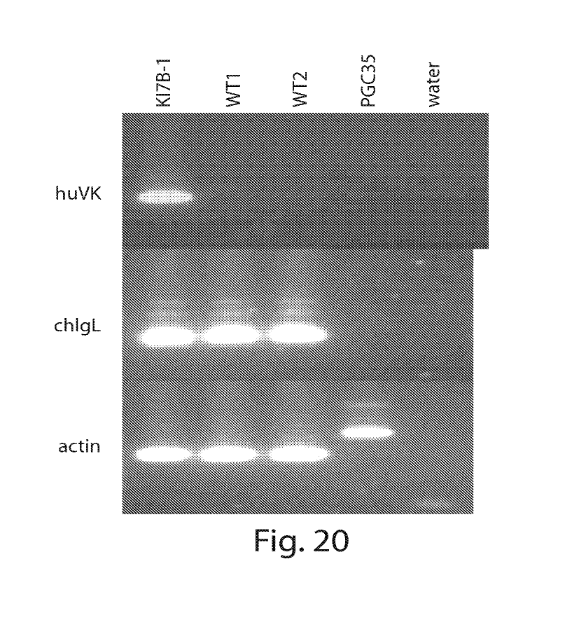

The correct size product was obtained for both sets of reactions (FIG. 20), and the chimeric IgL product was sequenced to verify its identity and to verify that the splice junctions between the human V leader exon, the human V coding exon, and the chicken C exon were correct. These results showed that the properly spliced transcript was produced and chimeric IgL was expressed in B lymphocytes of KI7B heterozygous chickens. Chicken IgL was also expressed because the chickens tested were KI7B heterozygous.

### Example 27

### KI/KO Chickens Express Human κ Light Chain

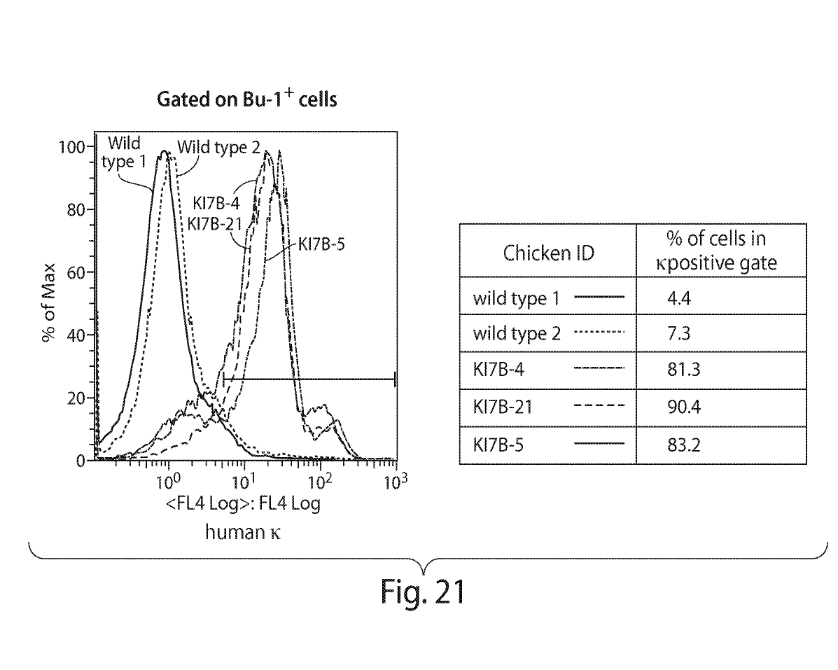

After establishing that KI/KO chickens produce B cells we wanted to assess if the chimeric light chain was expressed on the B cells. Expression of the chimeric light chain would require pairing with the chicken heavy chain to create the B cell receptor. Flow cytometry was used to if analyze expression of the human K light chain variable region. Lymphocytes isolated from the peripheral blood were stained with anti-chicken Bu-1 and anti-human κ light chain antibodies. Bu-1 positive cells were gated on and human κ light chain expression was assessed. B cells from the KI/KO chickens stained positive for human κ light chain whereas B cells from wild type chickens did not (FIG. 21). The two wild type chickens had low levels of kappa staining, likely background due to the polyclonal antibody used for staining. About 4 and 7 percent of the Bu-1+ cells in the wild type chickens were kappa positive. The majority of the Bu-1+ cells (ranging from 80-90 percent) in the IgLKI/KO chickens were kappa positive. This demonstrates that the human κ light chain is expressed on the B cells of the KI/KO chickens.

### Example 28

### Pairing of Chimeric chIgL-huVK Light Chain with Chicken Heavy Chain

We analyzed B cells by sacrificing a few G1 IgL KI chickens at hatching and verifying that B cells are present and expressing a cell surface IgM receptor containing the huVL region, to determine if the heavy and light chains were pairing properly. Bursas were dissected and ground with a syringe plunger against a wire mesh while bathed in PBS in a Petri dish to release the B cells inside. The minced tissues were collected in an Eppendorf tube and the debris allowed to settle. The cells in the supernatant were washed and resuspended in PBS with 1% FBS and layered over a Ficoll gradient and centrifuged. The B lymphocytes formed a layer at the interface, which were collected and washed several times to remove the Ficoll. Cells were then stained with anti-chicken IgM (μ-chain specific) mouse monoclonal antibodies (clone M-1; Southern Biotechnology Associates, Birmingham, Ala.); anti-Bu-1 antibodies (Bu-1 is a marker present on all B cells in the chicken); and antibodies against human kappa light chains. The antibodies were coupled to fluorophores such as fluorescein isothiocyanate (FITC), phycoerythrin (PE), and Cyanine™ 5 (Cy5)-PE for observation by fluorescent microscopy and FACS analysis. We did not expect to observe cells expressing both the endogenous light chain and the huVL light chain because such cells should be eliminated by apoptosis well before hatch. FIG. 21 shows the human κ light chain is expressed on Bu-1+B cells. As human κ light chain would not be present on the cell surface unless it was paired in an antibody with an endogenous chicken heavy chain, the staining indicates that the pairing has occurred.

### Example 29

### Analysis of G1 Chickens for Gene Conversion

Immunoglobulin diversity in chickens is generated by somatic gene conversion (GC) events in which sequences derived from upstream arrays of pseudogenes replace homologous sequences in unique and functionally rearranged immunoglobulin heavy and light chain variable regions. Because the functionally rearranged immunoglobulin light chain variable gene is human VK gene, it is important to analyze whether gene conversion occurs in the chIgL-huV transgenic chickens at different time points.

At hatching, bursal B cells expressing the huVL light chain were analyzed for GC to obtain a baseline level of GC. The huVL functional gene was PCR amplified from the B cell population and 100 cloned PCR products (which represent the huVL gene in 100 individual cells) were sequenced and aligned to the VK3-20 germline gene. In the wild type locus, a small amount of GC normally has occurred by hatching.

GC was assessed at 3 weeks after hatch. By that time, the endogenous light chain has undergone three to seven GC events in each B cell (Reynaud et al., Cell 48(3):379-88 (1987)). Peripheral blood was drawn at 34 days of age from the KI7B-1 chIgL-huV transgenic bird and total lymphocytes were prepared from K17B-1 and wild-type chickens by centrifugation over a Ficoll cushion. Total mRNA was extracted from the lymphocytes, reverse transcribed to cDNA, second strand cDNA produced, and PCR performed to amplify the human V region coding sequence. The upstream primer was in the human V leader exon (huVK3-20Sig-F) and the downstream primer was in the chicken IgL constant region (chCL-R) or in the human JK region (huJKrev). As a control, the endogenous actin mRNA was amplified, as described in Example 26.

Primer sequences for RT-PCR in chicken B cells:

huVK3-20Sig-F
(SEQ ID NO: 23)
GCTTCTCTTCCTCCTGCTACTCTG
chCL-R
(SEQ ID NO: 24)
TTCGTTCAGCTCCTCCTTTGACG
huJKrev
(SEQ ID NO: 25)
GTTTGATTTCCACCTTGGTCCC

AmpliTaq Gold® DNA Polymerase (Applied Biosystems, Foster City, Calif.) was used for PCR. After initial denaturation at 95° C. for 10 min, 35 cycles of PCR were performed by incubating the reaction mixture in the following conditions: 95° C. for 45 sec, 62° C. for 45 sec and 72° C. for 1 min. The PCR products were run on 1% agarose gel and gel purified via Qiaquick gel extraction kit (Qiagen Inc.). For each reaction, two PCR products (430 bp for huVK3-20Sig-F/chCL-R or 368 bp for huVK3-20Sig-F/huJKrev) were cloned by TOPO cloning and 184 independent clones were picked and sequenced with M13F and M13R primers.

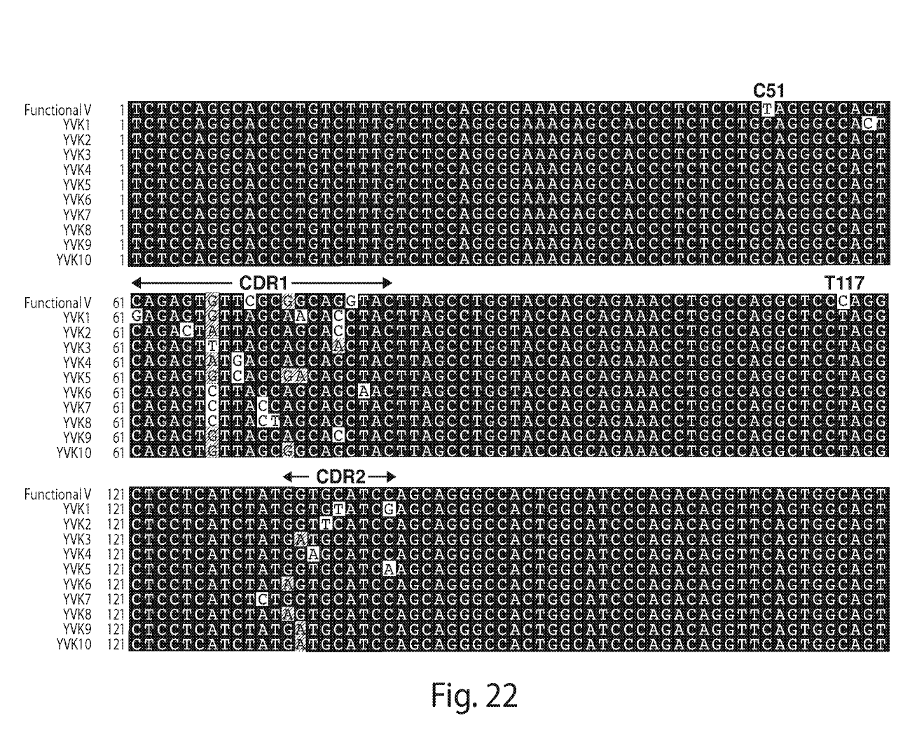

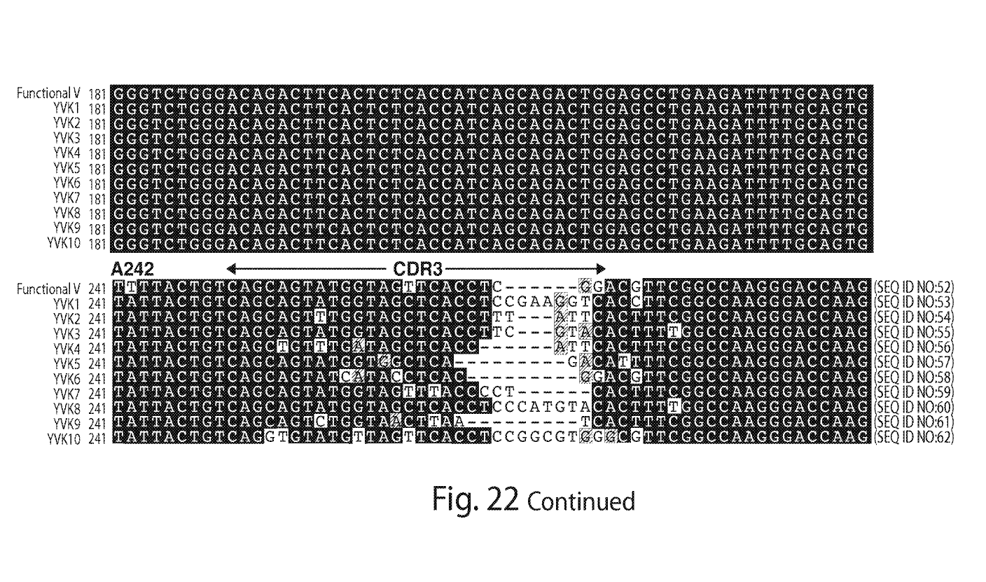

Categorization of gene conversion was performed by comparing the rate of mutations within CDRs to a baseline rate of mutation, which was determined by sequencing an irrelevant gene, the constant region of IgL, or the variable region of IgL in B cells. The baseline mutation rates were well below the observed mutation rates in this study, and analyses were conducted using Pfx Accuprime™ polymerase (Life Technologies™). Sequences were aligned using DNASTAR's MegAlign™ program (DNASTAR Inc., Madison, Wis.) High quality base discrepancies (bases with a quality exceeding a threshold that differs from a consensus sequence) were noted and subjected to further analysis. As the total mutation rate was much lower than 1 mutation/read, tracks of multiple mutations in a read were scored as gene conversion (GC) events. Single mutations for which no donor template could be identified were scored as point mutations. To categorize ambiguous mutations (which match the pseudogene templates but occur in isolation), results were compared when these mutations were excluded from the analysis, always considered point mutations, and always considered GC events. These changes made little difference to the final analysis, as the mutations in B cells very rarely matched the pseudogene sequences through either blast searches or direct comparison to a database of collected pseudogene sequences and so were able to be clearly scored as point mutations. To avoid missing any GC events which may occur in the B cells, it was decided to use the most inclusive definition of a GCV event, which is every mutation that matches the pseudogene sequences by blastn. Sequences were aligned with the input sequence (VK3-20) that was introduced on the huVK insertion vector, and CDR sequences were compared to the pseudogene CDRs. An alignment of the pseudogenes is shown in FIG. 22.

The sequences from the blood lymphocytes showed that gene conversion had modified the functional human V, using donor sequences from the upstream human pseudogene pool. All CDRs showed evidence of gene conversion, and all 10 pseudogenes were used in gene conversion of at least some of the sequences. Point mutations were also observed, in both the framework and CDR sequences. Table 5 shows a summary of the number of gene conversion events observed and the pseudogene usage.

TABLE 5
Each row shows the number of times a particular pseudogene was used
in a gene conversion event for each of the three CDRs of the functional
VK. Each column shows the number of gene conversion events for a
particular CDR. ND, not determined for some of the CDR2 sequences
because the pseudogenes 3, 6, 8, 9 and 10 are too similar to determine
which pseudogene was used. These numbers are likely an underestimate
because point mutations were not counted, even if the relevant base
pair could be identified in the pseudogene pool.

| | Number of | Number of | | |
|---|---|---|---|---|
| | times used in | times used in | Number of times | Total |
| Pseudogene | CDR1 | CDR2 | used in CDR3 | GC events |
| YVK1 | 3 | 7 | 0 | 10 |
| YVK2 | 8 | 12 | 1 | 21 |
| YVK3 | 13 | ND | 6 | >19 |
| YVK4 | 11 | 19 | 3 | 33 |
| YVK5 | 10 | 10 | 2 | 22 |
| YVK6 | 15 | ND | 8 | >23 |
| YVK7 | 9 | 16 | 2 | 27 |
| YVK8 | 12 | ND | 1 | >13 |
| YVK9 | 13 | ND | 5 | >18 |
| YVK10 | 13 | ND | 4 | >17 |
| Total GC | 107 | 64 | 32 | 203 |
| events | | | | |

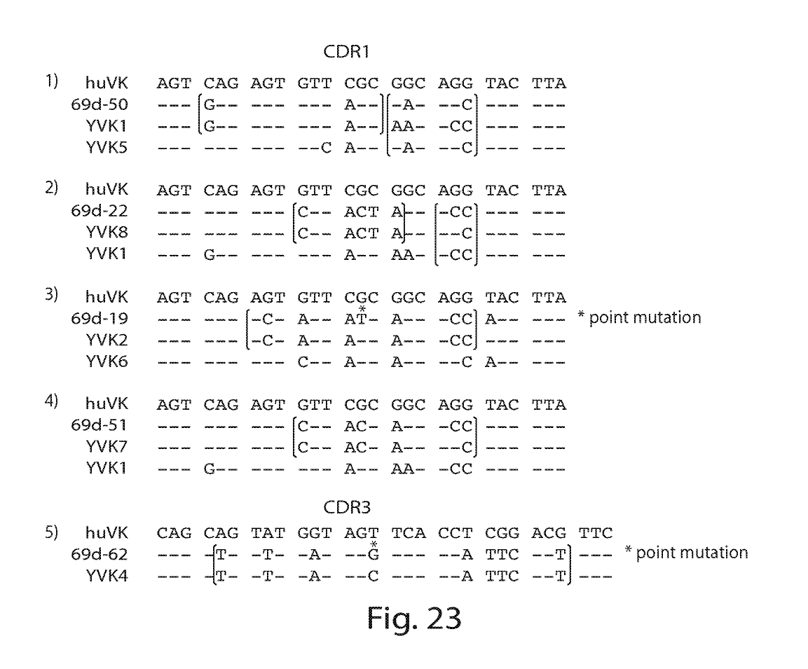

FIG. 23 shows five examples of gene conversion events, comparing the germline VK3-20 sequence to cloned B cell sequences and alignments with pseudogene donors. Many sequences showed multiple gene conversion events throughout the V region. Sometimes, a single sequence showed gene conversion by different pseudogenes, confirming independent gene conversion events. In other cases, gene conversion was observed but could not be assigned to a specific pseudogene, because the base pairs could have been donated by multiple pseudogenes. In those cases it was not always possible to determine the borders of the gene conversion event because the mutations could have been from one long gene conversion event or from multiple independent events. The minimum number of gene conversion events, averaged over all the sequences, was 1.8 events per sequence, and the maximum number (assuming all observed gene conversion events were independent events) was an average of 4.5 events per sequence. Sequences were compared at two time points, 34 days and 69 days after hatch, and the level of gene conversion was higher at 69 days, indicating that gene conversion is ongoing and cumulative in the B cell population.

In addition to the CDRs, there were three base pairs in the framework regions that were present in all the pseudogenes: C51 (a T residue in the germline VK3-20), T117 (a C residue in the germline VK3-20) and A242 (a T in the germline VK3-20). It was considered a gene conversion event if the base pair was observed to be identical to the pseudogene sequence, because although it is a point mutation, it is likely from gene conversion. The summary of mutation events in the compiled sequences is shown in Table 6.

TABLE 6
Summary of mutations in human V region sequences.
The six spots in the V regions where gene conversion
could be observed are shown in the six columns.

| | C51 | CDR1 | T117 | CDR2 | A242 | CDR3 |
|---|---|---|---|---|---|---|
| Number of | 167 | 161 | 168 | 165 | 166 | 159 |
| informative | | | | | | |
| sequences | | | | | | |
| GC | 120 | 118 | 115 | 118 | 128 | 32 |
| PM | 0 | 9 | 0 | 3 | 2 | 56 |
| Unmutated | 47 | 25 | 53 | 44 | 36 | 71 |

GC = gene conversion, PM = point mutation.

The gene conversion frequency appears to be lower in CDR3 than in CDR1 or 2 (Table 6). The J sequences present in the human pseudogenes downstream of CDR3 could affect the gene conversion frequency in CDR3. The endogenous pseudogenes do not contain J sequences. The point mutation frequency appears to be higher in CDR3 than elsewhere in the V region, which could be a result of the decreased gene conversion. Of 184 sequences, only 3 showed no evidence of any mutations.

### Example 30

### Immunization and Serum Collection

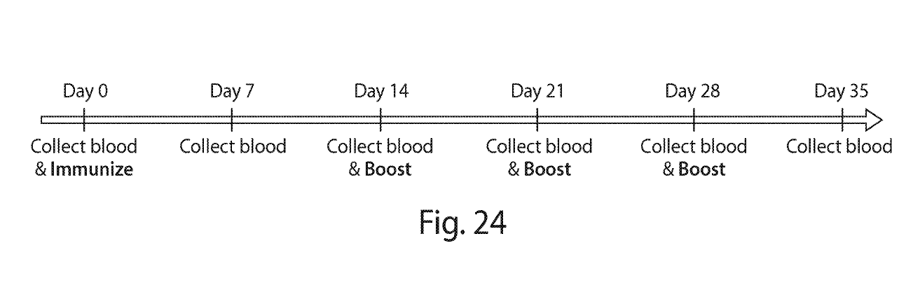

Approximately 500 μL of blood was collected from a wing vein of each chicken before initial vaccination with 250 μL of tetanus toxoid (Colorado Serum Company, Denver, Colo., concentration of toxoid unknown) in the back of the neck. One week later, 500 μL of blood was collected from each vaccinated chicken. Two weeks after the initial vaccination, a third 500 μL blood sample was collected from each immunized chicken and each chicken received a boost of 200 μL of tetanus toxoid vaccine. This process of blood sample collection and a boost in vaccination was repeated three and four weeks after the initial vaccination. The finial blood sample was collected 5 weeks after the initial immunization, with no boost given (FIG. 24). Serum was prepared from each blood sample by spinning the sample at high speed for 15 minutes at 4° C. The serum was removed from each sample and stored at −20° C.

### Example 31

### ELISA

The amount of tetanus-specific antibody was analyzed using a Tetanus ELISA kit (IBL International Corp., Toronto, Ontario). The kit protocol was followed. Briefly, samples were placed into wells of the microtiter plate from the kit. The plate was covered with foil and incubated for 1 hour at room temperature. The foil was then removed and the solution was discarded. The wells were then washed 3 times with 250 μL wash buffer (diluted 1:10 with water). Next, 250 μL of enzyme conjugate was added to each well. The plate was covered in foil and incubated for 1 hour at room temperature. The foil was removed and the solution was discarded. The plate was again washed 3 times with diluted wash buffer. Two hundred μL of 3,3′,5,5′-tetramethylbenzidine (TMB) substrate solution was added to each well, which were then incubated for 30 minutes at room temperature. Finally, 50 μL of TMB stop solution was added to each well. The OD was read using a SpectraMax 384 Plus (Molecular Devices, Inc., Sunnyvale, Calif.).

### Example 32

### Production of Antigen-Specific Antibodies in Serum of Hyperimmunized Transgenic Chickens

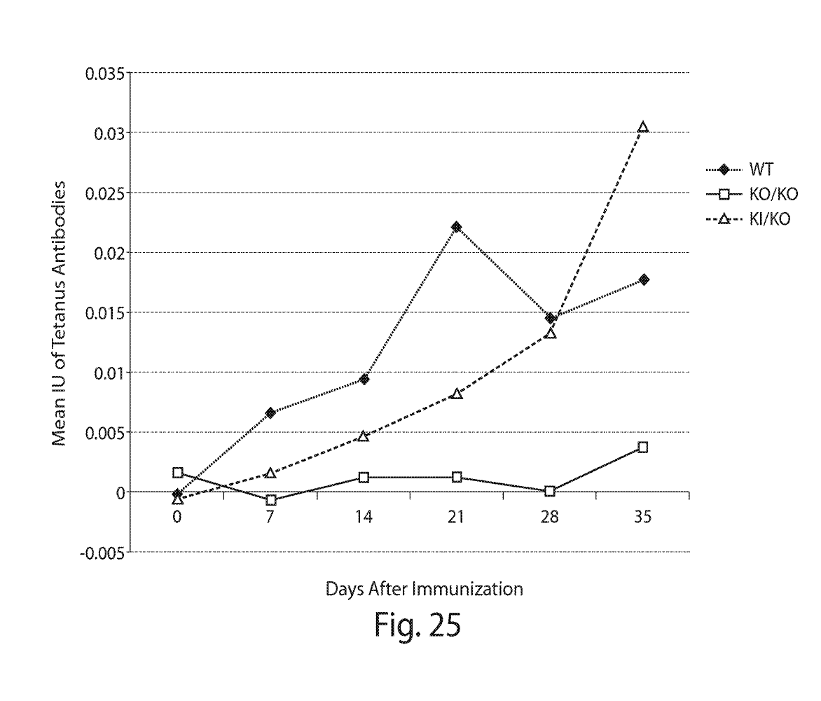

To test if the B cell receptor containing the chimeric light chain is functional, an immunization experiment was carried out. Following immunization procedures, serum was isolated from the blood samples and ELISA was used to detect tetanus-specific antibodies. As expected, since IgLKO/KO chickens do not produce B cells, IgLKO/KO birds were unable to produce significant amounts of tetanus specific antibodies. The highest average titer of tetanus specific antibodies from the IgLKO/KO chickens was 0.275 IU, seen 35 days after the initial immunization and after 3 boosts. This is lower than the average titer seen in wild type birds after the first immunization and no boosts, which was 0.363 IU. The IgLKI/KO chickens were able to produce tetanus specific antibodies at levels similar to the wild type chickens (FIG. 25). Early in the response, 7, 12, and 21 days after the initial immunization, the average titer of tetanus-specific antibodies was lower in IgLKI/KO than in wild type chickens. However at 28 days after the initial immunizations, the average titer in IgLKI/KO chickens was very similar to wild type (0.59 IU and 0.56 IU for IgLKI/KO and wild type respectively. By 35 days after the initial immunization and after 3 boosts, the average titer of tetanus specific antibody in IgLKI/KO was greater than the average titer in the wild type chickens (1.1 IU for IgLKI/KOverses 0.69 IU for wild type). This demonstrates that the human K light chain can serve as a component of a functional and antigen specific B cell receptor.

### Example 33

### Sequences of Human VK Pseudogenes

Spacer sequences are in upper case, restriction sites are in italics, and the VK sequence is in lower case. The loxP site adjacent to YVK10 is underlined. (Note: the sequences below are the full sequences of the functional VK3-20 gene and of pseudogenes (“YVK”) 1-10. FIG. 22 shows an alignment of each of these sequences. The sequences set forth in the Figure exclude a few nucleotides on the 5′ or '3 side of the alignment to focus attention on the positions in which substitutions or other differences exist.)

YVK1
(SEQ ID NO: 26)
agatctCTGTGCCCGCAGTCACATGTGGAATATCAAGACACACACATCTATGACAAtc
tccaggcaccctgtctttgtctccaggggaaagagccaccctctcctgcagggccactgagagtgttagcaacacctacttagcctggt
accagcagaaacctggccaggctcctaggctcctcatctatggtgtatcgagcagggccactggcatcccagacaggttcagtggca
gtgggtctgggacagacttcactctcaccatcagcagactggagcctgaagattagcagtgtattactgtcagcagtatggtagctcac
ctccgaaggtcaccttcggccaagggaccaag gtggaaatcaaaggatcc
YVK2
(SEQ ID NO: 27)
Ggatcctctccaggcaccctgtctagtctccaggggaaagagccaccctctcctgcagggccagtcagactattagcagcacctactt
agcctggtaccagcagaaacctggccaggctcctaggctcctcatctatggttcatccagcagggccactggcatcccagacaggttc
agtggcagtgggtctgggacagacttcactctcaccatcagcagactggagcctgaagattttgcagtgtattactgtcagcagtttggt
agctcacctttattcactttcggccaagggaccaaggtggaaatcaaaACCATGGATAGAGCTGGGAGCCCTC
ACTGCCACTCATGCCTTCAGGTGTCagatct
YVK3
(SEQ ID NO: 28)
agatctTGTAGTGAGCAGGGAGAGCACTGCAATAGGAGCTGATAGTGATCACACAGt
ctccaggcaccctgtctttgtctccaggggaaagagccaccctctcctgcagggccagtcagagttttagcagcaactacttagcctgg
taccagcagaaacctggccaggctcctaggctcctcatctatgatgcatccagcagggccactggcatcccagacaggttcagtggc
agtgggtctgggacagacttcactctcaccatcagcagactggagcctgaagattttgcagtgtattactgtcagcagtatggtagctca
ccttcgtacacttttggccaagggaccaaggtggaaatcaaaggatcc
YVK4
(SEQ ID NO: 29)
Ggatcctctccaggcaccctgtctagtctccaggggaaagagccaccctctcctgcagggccagtcagagtatgagcagcagctac
ttagcctggtaccagcagaaacctggccaggctcctaggctcctcatctatggagcatccagcagggccactggcatcccagacagg
ttcagtggcagtgggtctgggacagacttcactctcaccatcagcagactggagcctgaagattttgcagtgtattactgtcagctgtttg
atagctcaccattcactacggccaagggaccaaggtggaaatcaaaAGTCGGTGTTTGAATATTCTGTGTGT
GCTTGTGTGCTCTGGGGTCTCCTCagatct
YVK5
(SEQ ID NO: 30)
agatctAGGCAGACAGAAACCTGTCATTTTTAGCTCTAGACATCACATCACTCCCAtct
ccaggcaccctgtctttgtctccaggggaaagagccaccctctcctgcagggccagtcagagtgtcagcgacagctacttagcctggt
accagcagaaacctggccaggctcctaggctcctcatctatggtgcatcaagcagggccactggcatcccagacaggttcagtggca
gtgggtctgggacagacttcactctcaccatcagcagactggagcctgaagattagcagtgtattactgtcagcagtatggtggctcag
acattttcggccaagggaccaaggtggaaatcaaaggatcc
YVK6
(SEQ ID NO: 31)
Ggatcctctccaggcaccctgtctagtctccaggggaaagagccaccctctcctgcagggccagtcagagtcttagcagcagcaact
tagcctggtaccagcagaaacctggccaggctcctaggctcctcatctatagtgcatccagcagggccactggcatcccagacaggtt
cagtggcagtgggtctgggacagacttcactctcaccatcagcagactggagcctgaagattttgcagtgtattactgtcagcagtatca
tacctcacggacgttcggccaagggaccaaggtggaaatcaaaTGCTGGTATAGAGACAAAAGAGGATGT
GGAACTGAGTTACAGACCTGAGTagatct
YVK7
(SEQ ID NO: 32)
agatctTATCTCCCTGTGTCTCTGTACCTACAAAACTGCTGTCATAGGCCCCACTAtctc
caggcaccctgtctttgtctccaggggaaagagccaccctctcctgcagggccagtcagagtcttaccagcagctacttagcctggtac
cagcagaaacctggccaggctcctaggctcctcatctctggtgcatccagcagggccactggcatcccagacaggttcagtggcagt
gggtctgggacagacttcactctcaccatcagcagactggagcctgaagattttgcagtgtattactgtcagcagtatggtagtttacccc
tcactttcggccaagggaccaaggtggaaatcaaaggatcc
YVK8
(SEQ ID NO: 33)
Ggatcctctccaggcaccctgtctagtctccaggggaaagagccaccctctcctgcagggccagtcagagtcttactagcagctactt
agcctggtaccagcagaaacctggccaggctcctaggctcctcatctatagtgcatccagcagggccactggcatcccagacaggtt
cagtggcagtgggtctgggacagacttcactctcaccatcagcagactggagcctgaagattttgcagtgtattactgtcagcagtatgg
tagctcacctcccatgtacacttttggccaagggaccaaggtggaaatcaaaTCTGGGGTAACAGTCAAGGTCTT
GGCCATTCAGATAGGACAAGGCCTCCTagatct
YVK9
(SEQ ID NO: 34)
agatctACTGTCACACTAACTACCACTGTGGTCTAAGCTGTGGAGAACACTGCCCAtct
ccaggcaccctgtctttgtctccaggggaaagagccaccctctcctgcagggccagtcagagtgttagcagcacctacttagcctggt
accagcagaaacctggccaggctcctaggctcctcatctatgatgcatccagcagggccactggcatcccagacaggttcagtggca
gtgggtctgggacagacttcactctcaccatcagcagactggagcctgaagattagcagtgtattactgtcagcagtctggtaacttaat
cactttcggccaagggaccaaggtggaaatcaaaggatcc
YVK10
(SEQ ID NO: 35)
ggatccccaggcaccctgtctttgtctccaggggaaagagccaccctctcctgcagggccagtcagagtgttagcggcag
ctacttagcctggtaccagcagaaacctggccaggctcctaggctcctcatctatgatgcatccagcagggccactggca
tcccagacaggttcagtggcagtgggtctgggacagacttcactctcaccatcagcagactggagcctgaagattttgca
gtgtattactgtcaggtgtatgttagttcacctccggcgtgggcgttcggccaagggaccaaggtggaaatcaaaagatc
tATAACTTCGTATAATGTATGCTATACGAAGTTATgcggccgc

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

We claim:

1.-15. (canceled)

16. An isolated nucleic acid construct comprising, in the following order, read 5′ to 3′:
(a) a plurality of human or humanized pseudogenes, wherein said pseudogenes comprise a nucleotide sequence of from 20 nucleotides to about 1000 nucleotides, wherein the plurality of pseudogenes are arranged in direct repeat orientation, optionally wherein said sequence encodes at least a portion of a human or a humanized VL chain,
(b) a promoter operative in an avian B cell, and
(c) a variable region segment encoding a variable region of a human or humanized immunoglobulin kappa light chain (HuVL),
wherein the promoter is operatively linked with the variable region segment and wherein each pseudogene has sufficient homology to the segment encoding HuVL to permit gene conversion when the construct is present in an avian immunoglobulin light chain locus in an avian B cell during B cell maturation.

17. The nucleic acid construct of claim 16, further comprising:
(d) a nucleic acid sequence encoding a human, humanized, or avian constant region.

18. The nucleic acid construct of claim 16, further wherein said avian B cell of (b) is a chicken B cell and wherein each pseudogene has sufficient homology to the segment encoding HuVL to permit gene conversion when the construct is present in a chicken immunoglobulin light chain locus in a chicken B cell during the chicken B cell maturation.

19. The nucleic acid construct of claim 16, further wherein said HuVL is a human immunoglobulin kappa light chain variable region.

20. A targeting vector comprising a nucleic acid construct of claim 16.

21. (canceled)

22. An expression cassette comprising a nucleic acid construct of claim 16.

23. An insertion vector, said vector comprising a nucleic acid construct of claim 16 and an insertion sequence permitting insertion into an avian immunoglobulin light chain gene in an avian cell.

24. The insertion vector of claim 23, wherein said insertion sequence comprises an attP sequence.

25. The insertion vector of claim 23, wherein said avian immunoglobulin light chain gene and said avian cell are from Gallus gallus domesticus.

26. A recombinant avian chromosome comprising the nucleic acid construct of claim 16.

27.-28. (canceled)

29. An avian cell comprising a nucleic acid construct of claim 16.

30.-33. (canceled)

34. A transgenic bird comprising a nucleic acid construct of claim 16.

35.-48. (canceled)

49. A method of making polyclonal antibodies specific for a target antigen, said method comprising contacting a bird of claim 34 with said target antigen.

50.-53. (canceled)

54. A chicken cell line comprising a nucleic acid construct of claim 16, which produces monoclonal antibodies, which antibodies comprise a humanized variable region.

55. A method of making a transgenic bird comprising:
a) knocking out a bird immunoglobulin gene in a primordial germ cell of the bird;
b) inserting into the knocked out immunoglobulin gene:
(i) a plurality of human or humanized pseudogenes, each comprising a nucleotide sequence of from 20 nucleotides to about 1000 nucleotides, wherein the nucleotide sequence optionally encodes at least a portion of a human or a humanized VL chain, wherein said pseudogene is under control of a promoter operative in a B cell of said avian, wherein the plurality of pseudogenes are arranged in direct repeat orientation;
(ii) at least one human or humanized immunoglobulin gene segment selected from the group consisting of a Variable immunoglobulin gene segment, and a Joining immunoglobulin gene segment, and
(iii) a segment encoding a human or a chicken constant region, thereby creating a transgenic primordial germ cell;
c) introducing said transgenic primordial germ cell into a bird embryo; and
d) growing said bird embryo into an adult bird such that said transgenic germ cell integrates into a germline of said embryo.

56. A method of claim 55, wherein said bird is a Galliformes.

57. A method of claim 56, wherein said Gallifomes is of the species Gallus gallus.

58. A method comprising:
a) collecting an egg laid by a bird of claim 34, wherein said egg comprises polyclonal antibodies produced by said bird; and
b) isolating said polyclonal antibodies.

59. The nucleic acid construct of claim 16, wherein each pseudogene has sufficient homology to the segment encoding HuVL to permit at least 10 gene conversion (GC) events.

60. The nucleic acid construct of claim 16, which comprises 26-65 pseudogenes.


SEQUENCE LISTING (79 sequences)

SEQ ID NO: 1 - DNA, 23 bp, Artificial Sequence
  Description of Artificial Sequence Synthetic primer
agaccctcggacatcccttcacg

SEQ ID NO: 2 - DNA, 24 bp, Artificial Sequence
  Description of Artificial Sequence Synthetic primer
aaaacccccaaatcaccaaaaatc

SEQ ID NO: 3 - DNA, 22 bp, Artificial Sequence
  Description of Artificial Sequence Synthetic primer
acgacagacttgaggggttctc

SEQ ID NO: 4 - DNA, 20 bp, Artificial Sequence
  Description of Artificial Sequence Synthetic primer
gctcttcagcaatatcacgg

SEQ ID NO: 5 - DNA, 25 bp, Artificial Sequence
  Description of Artificial Sequence Synthetic primer
aggatcctgtagagcctcaggactg

SEQ ID NO: 6 - DNA, 21 bp, Artificial Sequence
  Description of Artificial Sequence Synthetic primer
gaactctctatgaccatggcc

SEQ ID NO: 7 - DNA, 23 bp, Artificial Sequence
  Description of Artificial Sequence Synthetic primer
agaccctcggacatcccttcacg

SEQ ID NO: 8 - DNA, 24 bp, Artificial Sequence
  Description of Artificial Sequence Synthetic primer
aaaacccccaaatcaccaaaaatc

SEQ ID NO: 9 - DNA, 22 bp, Artificial Sequence
  Description of Artificial Sequence Synthetic primer
acgacagacttgaggggttctc

SEQ ID NO: 10 - DNA, 20 bp, Artificial Sequence
  Description of Artificial Sequence Synthetic primer
gctcttcagcaatatcacgg

SEQ ID NO: 11 - DNA, 24 bp, Artificial Sequence
  Description of Artificial Sequence Synthetic primer
gcttctcttcctcctgctactctg

SEQ ID NO: 12 - DNA, 24 bp, Artificial Sequence
  Description of Artificial Sequence Synthetic primer
aaaacccccaaatcaccaaaaatc

SEQ ID NO: 13 - DNA, 24 bp, Artificial Sequence
  Description of Artificial Sequence Synthetic primer
gcttctcttcctcctgctactctg

SEQ ID NO: 14 - DNA, 23 bp, Artificial Sequence
  Description of Artificial Sequence Synthetic primer
ttcgttcagctcctcctttgacg

SEQ ID NO: 15 - DNA, 22 bp, Artificial Sequence
  Description of Artificial Sequence Synthetic primer
gtttgatttccaccttggtccc

SEQ ID NO: 16 - DNA, 23 bp, Artificial Sequence
  Description of Artificial Sequence Synthetic primer
agaaagaccgagacgaggtcagc

SEQ ID NO: 17 - DNA, 21 bp, Artificial Sequence
  Description of Artificial Sequence Synthetic primer
aacaccccagccatgtatgta

SEQ ID NO: 18 - DNA, 20 bp, Artificial Sequence
  Description of Artificial Sequence Synthetic primer
tttcattgtgctaggtgcca

SEQ ID NO: 19 - DNA, 24 bp, Artificial Sequence
  Description of Artificial Sequence Synthetic primer
gcttctcttcctcctgctactctg

SEQ ID NO: 20 - DNA, 23 bp, Artificial Sequence
  Description of Artificial Sequence Synthetic primer
ttcgttcagctcctcctttgacg

SEQ ID NO: 21 - DNA, 21 bp, Artificial Sequence
  Description of Artificial Sequence Synthetic primer
aacaccccagccatgtatgta

SEQ ID NO: 22 - DNA, 20 bp, Artificial Sequence
  Description of Artificial Sequence Synthetic primer
tttcattgtgctaggtgcca

SEQ ID NO: 23 - DNA, 24 bp, Artificial Sequence
  Description of Artificial Sequence Synthetic primer
gcttctcttcctcctgctactctg

SEQ ID NO: 24 - DNA, 23 bp, Artificial Sequence
  Description of Artificial Sequence Synthetic primer
ttcgttcagctcctcctttgacg

SEQ ID NO: 25 - DNA, 22 bp, Artificial Sequence
  Description of Artificial Sequence Synthetic primer
gtttgatttccaccttggtccc

SEQ ID NO: 26 - DNA, 374 bp, Homo sapiens
agatctctgtgcccgcagtcacatgtggaatatcaagacacacacatctatgacaatctccaggcaccctgtctttgtctccaggggaaagagccaccctctcctgcagggccactgagagtgttagcaacacctacttagcctggtaccagcagaaacctggccaggctcctaggctcctcatctatggtgtatcgagcagggccactggcatcccagacaggttcagtggcagtgggtctgggacagacttcactctcaccatcagcagactggagcctgaagattttgcagtgtattactgtcagcagtatggtagctcacctccgaaggtcaccttcggccaagggaccaaggtggaaatcaaaggatcc

SEQ ID NO: 27 - DNA, 371 bp, Homo sapiens
ggatcctctccaggcaccctgtctttgtctccaggggaaagagccaccctctcctgcagggccagtcagactattagcagcacctacttagcctggtaccagcagaaacctggccaggctcctaggctcctcatctatggttcatccagcagggccactggcatcccagacaggttcagtggcagtgggtctgggacagacttcactctcaccatcagcagactggagcctgaagattttgcagtgtattactgtcagcagtttggtagctcacctttattcactttcggccaagggaccaaggtggaaatcaaaaccatggatagagctgggagccctcactgccactcatgccttcaggtgtcagatct

SEQ ID NO: 28 - DNA, 371 bp, Homo sapiens
agatcttgtagtgagcagggagagcactgcaataggagctgatagtgatcacacagtctccaggcaccctgtctttgtctccaggggaaagagccaccctctcctgcagggccagtcagagttttagcagcaactacttagcctggtaccagcagaaacctggccaggctcctaggctcctcatctatgatgcatccagcagggccactggcatcccagacaggttcagtggcagtgggtctgggacagacttcactctcaccatcagcagactggagcctgaagattttgcagtgtattactgtcagcagtatggtagctcaccttcgtacacttttggccaagggaccaaggtggaaatcaaaggatcc

SEQ ID NO: 29 - DNA, 368 bp, Homo sapiens
ggatcctctccaggcaccctgtctttgtctccaggggaaagagccaccctctcctgcagggccagtcagagtatgagcagcagctacttagcctggtaccagcagaaacctggccaggctcctaggctcctcatctatggagcatccagcagggccactggcatcccagacaggttcagtggcagtgggtctgggacagacttcactctcaccatcagcagactggagcctgaagattttgcagtgtattactgtcagctgtttgatagctcaccattcactttcggccaagggaccaaggtggaaatcaaaagtcggtgtttgaatattctgtgtgtgcttgtgtgctctggggtctcctcagatct

SEQ ID NO: 30 - DNA, 365 bp, Homo sapiens
agatctaggcagacagaaacctgtcatttttagctctagacatcacatcactcccatctccaggcaccctgtctttgtctccaggggaaagagccaccctctcctgcagggccagtcagagtgtcagcgacagctacttagcctggtaccagcagaaacctggccaggctcctaggctcctcatctatggtgcatcaagcagggccactggcatcccagacaggttcagtggcagtgggtctgggacagacttcactctcaccatcagcagactggagcctgaagattttgcagtgtattactgtcagcagtatggtggctcagacattttcggccaagggaccaaggtggaaatcaaaggatcc

SEQ ID NO: 31 - DNA, 365 bp, Homo sapiens
ggatcctctccaggcaccctgtctttgtctccaggggaaagagccaccctctcctgcagggccagtcagagtcttagcagcagcaacttagcctggtaccagcagaaacctggccaggctcctaggctcctcatctatagtgcatccagcagggccactggcatcccagacaggttcagtggcagtgggtctgggacagacttcactctcaccatcagcagactggagcctgaagattttgcagtgtattactgtcagcagtatcatacctcacggacgttcggccaagggaccaaggtggaaatcaaatgctggtatagagacaaaagaggatgtggaactgagttacagacctgagtagatct

SEQ ID NO: 32 - DNA, 368 bp, Homo sapiens
agatcttatctccctgtgtctctgtacctacaaaactgctgtcataggccccactatctccaggcaccctgtctttgtctccaggggaaagagccaccctctcctgcagggccagtcagagtcttaccagcagctacttagcctggtaccagcagaaacctggccaggctcctaggctcctcatctctggtgcatccagcagggccactggcatcccagacaggttcagtggcagtgggtctgggacagacttcactctcaccatcagcagactggagcctgaagattttgcagtgtattactgtcagcagtatggtagtttacccctcactttcggccaagggaccaaggtggaaatcaaaggatcc

SEQ ID NO: 33 - DNA, 374 bp, Homo sapiens
ggatcctctccaggcaccctgtctttgtctccaggggaaagagccaccctctcctgcagggccagtcagagtcttactagcagctacttagcctggtaccagcagaaacctggccaggctcctaggctcctcatctatagtgcatccagcagggccactggcatcccagacaggttcagtggcagtgggtctgggacagacttcactctcaccatcagcagactggagcctgaagattttgcagtgtattactgtcagcagtatggtagctcacctcccatgtacacttttggccaagggaccaaggtggaaatcaaatctggggtaacagtcaaggtcttggccattcagataggacaaggcctcctagatct

SEQ ID NO: 34 - DNA, 365 bp, Homo sapiens
agatctactgtcacactaactaccactgtggtctaagctgtggagaacactgcccatctccaggcaccctgtctttgtctccaggggaaagagccaccctctcctgcagggccagtcagagtgttagcagcacctacttagcctggtaccagcagaaacctggccaggctcctaggctcctcatctatgatgcatccagcagggccactggcatcccagacaggttcagtggcagtgggtctgggacagacttcactctcaccatcagcagactggagcctgaagattttgcagtgtattactgtcagcagtctggtaacttaatcactttcggccaagggaccaaggtggaaatcaaaggatcc

SEQ ID NO: 35 - DNA, 363 bp, Homo sapiens
ggatccccaggcaccctgtctttgtctccaggggaaagagccaccctctcctgcagggccagtcagagtgttagcggcagctacttagcctggtaccagcagaaacctggccaggctcctaggctcctcatctatgatgcatccagcagggccactggcatcccagacaggttcagtggcagtgggtctgggacagacttcactctcaccatcagcagactggagcctgaagattttgcagtgtattactgtcaggtgtatgttagttcacctccggcgtgggcgttcggccaagggaccaaggtggaaatcaaaagatctataacttcgtataatgtatgctatacgaagttatgcggccgc

SEQ ID NO: 36 - DNA, 384 bp, Homo sapiens
atggaaaccccagcgcagcttctcttcctcctgctactctggctcccagataccaccggagaaattgtgttgacgcagtctccaggcaccctgtctttgtctccaggggaaagagccaccctctcctgtagggccagtcagagtgttcgcggcaggtacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgttttactgtcagcagtatggtagttcacctcggacgttcggccaagggaccaaggtggaaatcaaa

SEQ ID NO: 37 - protein, 108 aa, Homo sapiens
EIVLTQSPGTLSLSPGERATLSCRASQSVRGRYLAWYQQKPGQAPRLLIYGASSRATGIPDRFSGSGSGTDFTLTISRLEPEDFAVFYCQQYGSSPRTFGQGTKVEIK

SEQ ID NO: 38 - protein, 108 aa, Homo sapiens
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPWTFGQGTKVEIK

SEQ ID NO: 39 - DNA, 348 bp, Homo sapiens
atggaaaccccagcgcagcttctcttcctcctgctactctggctcccagataccaccggagaaattgtgttgacgcagtctccaggcaccctgtctttgtctccaggggaaagagccaccctctcctgtagggccagtcagagtgttcgcggcaggtacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgttttactgtcagcagtatggtagttcacct

SEQ ID NO: 40 - DNA, 473 bp, Homo sapiens
atggaaaccccagcgcagcttctcttcctcctgctactctggctcccaggtactcgttgcgcccggtcggggactgtgggcacggggctctgtcccactgctgcgcgggcagggctgtgcgtgcggggccgtcactgattgccgttttctcccctctctcctctccctctccagataccaccggagaaattgtgttgacgcagtctccaggcaccctgtctttgtctccaggggaaagagccaccctctcctgtagggccagtcagagtgttcgcggcaggtacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgttttactgtcagcagtatggtagttcacct

SEQ ID NO: 41 - protein, 102 aa, Homo sapiens
SPGTLSLSPGERATLSCRASQSVSSSYLAWYQQKPGQAPRLLIYGASSRATGIPDRFSGSGSGTDFTLTISRLEPEDFAVYYCQQYGSSPWTFGQGTKVEIK

SEQ ID NO: 42 - protein, 104 aa, Homo sapiens
SPGTLSLSPGERATLSCRATESVSNTYLAWYQQKPGQAPRLLIYGVSSRATGIPDRFSGSGSGTDFTLTISRLEPEDFAVYYCQQYGSSPPKVTFGQGTKVEIK

SEQ ID NO: 43 - protein, 103 aa, Homo sapiens
SPGTLSLSPGERATLSCRASQTISSTYLAWYQQKPGQAPRLLIYGSSSRATGIPDRFSGSGSGTDFTLTISRLEPEDFAVYYCQQFGSSPLFTFGQGTKVEIK

SEQ ID NO: 44 - protein, 103 aa, Homo sapiens
SPGTLSLSPGERATLSCRASQSFSSNYLAWYQQKPGQAPRLLIYDASSRATGIPDRFSGSGSGTDFTLTISRLEPEDFAVYYCQQYGSSPSYTFGQGTKVEIK

SEQ ID NO: 45 - protein, 102 aa, Homo sapiens
SPGTLSLSPGERATLSCRASQSMSSSYLAWYQQKPGQAPRLLIYGASSRATGIPDRFSGSGSGTDFTLTISRLEPEDFAVYYCQLFDSSPFTFGQGTKVEIK

SEQ ID NO: 46 - protein, 101 aa, Homo sapiens
SPGTLSLSPGERATLSCRASQSVSDSYLAWYQQKPGQAPRLLIYGASSRATGIPDRFSGSGSGTDFTLTISRLEPEDFAVYYCQQYGGSDIFGQGTKVEIK

SEQ ID NO: 47 - protein, 101 aa, Homo sapiens
SPGTLSLSPGERATLSCRASQSLSSSNLAWYQQKPGQAPRLLIYSASSRATGIPDRFSGSGSGTDFTLTISRLEPEDFAVYYCQQYHTSRTFGQGTKVEIK

SEQ ID NO: 48 - protein, 102 aa, Homo sapiens
SPGTLSLSPGERATLSCRASQSLTSSYLAWYQQKPGQAPRLLISGASSRATGIPDRFSGSGSGTDFTLTISRLEPEDFAVYYCQQYGSLPLTFGQGTKVEIK

SEQ ID NO: 49 - protein, 104 aa, Homo sapiens
SPGTLSLSPGERATLSCRASQSLTSSYLAWYQQKPGQAPRLLIYSASSRATGIPDRFSGSGSGTDFTLTISRLEPEDFAVYYCQQYGSSPPMYTFGQGTKVEIK

SEQ ID NO: 50 - protein, 101 aa, Homo sapiens
SPGTLSLSPGERATLSCRASQSVSSTYLAWYQQKPGQAPRLLIYDASSRATGIPDRFSGSGSGTDFTLTISRLEPEDFAVYYCQQSGNLITFGQGTKVEIK

SEQ ID NO: 51 - protein, 104 aa, Homo sapiens
SPGTLSLSPGERATLSCRASQSVSGSYLAWYQQKPGQAPRLLIYDASSRATGIPDRFSGSGSGTDFTLTISRLEPEDFAVYYCQVYVSSPPAWAFGQGTKVEIK

SEQ ID NO: 52 - DNA, 294 bp, Homo sapiens
tctccaggcaccctgtctttgtctccaggggaaagagccaccctctcctgtagggccagtcagagtgttcgcggcaggtacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgttttactgtcagcagtatggtagttcacctcggacgttcggccaagggaccaag

SEQ ID NO: 53 - DNA, 300 bp, Homo sapiens
tctccaggcaccctgtctttgtctccaggggaaagagccaccctctcctgcagggccactgagagtgttagcaacacctacttagcctggtaccagcagaaacctggccaggctcctaggctcctcatctatggtgtatcgagcagggccactggcatcccagacaggttcagtggcagtgggtctgggacagacttcactctcaccatcagcagactggagcctgaagattttgcagtgtattactgtcagcagtatggtagctcacctccgaaggtcaccttcggccaagggaccaag

SEQ ID NO: 54 - DNA, 297 bp, Homo sapiens
tctccaggcaccctgtctttgtctccaggggaaagagccaccctctcctgcagggccagtcagactattagcagcacctacttagcctggtaccagcagaaacctggccaggctcctaggctcctcatctatggttcatccagcagggccactggcatcccagacaggttcagtggcagtgggtctgggacagacttcactctcaccatcagcagactggagcctgaagattttgcagtgtattactgtcagcagtttggtagctcacctttattcactttcggccaagggaccaag

SEQ ID NO: 55 - DNA, 297 bp, Homo sapiens
tctccaggcaccctgtctttgtctccaggggaaagagccaccctctcctgcagggccagtcagagttttagcagcaactacttagcctggtaccagcagaaacctggccaggctcctaggctcctcatctatgatgcatccagcagggccactggcatcccagacaggttcagtggcagtgggtctgggacagacttcactctcaccatcagcagactggagcctgaagattttgcagtgtattactgtcagcagtatggtagctcaccttcgtacacttttggccaagggaccaag

SEQ ID NO: 56 - DNA, 294 bp, Homo sapiens
tctccaggcaccctgtctttgtctccaggggaaagagccaccctctcctgcagggccagtcagagtatgagcagcagctacttagcctggtaccagcagaaacctggccaggctcctaggctcctcatctatggagcatccagcagggccactggcatcccagacaggttcagtggcagtgggtctgggacagacttcactctcaccatcagcagactggagcctgaagattttgcagtgtattactgtcagctgtttgatagctcaccattcactttcggccaagggaccaag

SEQ ID NO: 57 - DNA, 291 bp, Homo sapiens
tctccaggcaccctgtctttgtctccaggggaaagagccaccctctcctgcagggccagtcagagtgtcagcgacagctacttagcctggtaccagcagaaacctggccaggctcctaggctcctcatctatggtgcatcaagcagggccactggcatcccagacaggttcagtggcagtgggtctgggacagacttcactctcaccatcagcagactggagcctgaagattttgcagtgtattactgtcagcagtatggtggctcagacattttcggccaagggaccaag

SEQ ID NO: 58 - DNA, 291 bp, Homo sapiens
tctccaggcaccctgtctttgtctccaggggaaagagccaccctctcctgcagggccagtcagagtcttagcagcagcaacttagcctggtaccagcagaaacctggccaggctcctaggctcctcatctatagtgcatccagcagggccactggcatcccagacaggttcagtggcagtgggtctgggacagacttcactctcaccatcagcagactggagcctgaagattttgcagtgtattactgtcagcagtatcatacctcacggacgttcggccaagggaccaag

SEQ ID NO: 59 - DNA, 294 bp, Homo sapiens
tctccaggcaccctgtctttgtctccaggggaaagagccaccctctcctgcagggccagtcagagtcttaccagcagctacttagcctggtaccagcagaaacctggccaggctcctaggctcctcatctctggtgcatccagcagggccactggcatcccagacaggttcagtggcagtgggtctgggacagacttcactctcaccatcagcagactggagcctgaagattttgcagtgtattactgtcagcagtatggtagtttacccctcactttcggccaagggaccaag

SEQ ID NO: 60 - DNA, 300 bp, Homo sapiens
tctccaggcaccctgtctttgtctccaggggaaagagccaccctctcctgcagggccagtcagagtcttactagcagctacttagcctggtaccagcagaaacctggccaggctcctaggctcctcatctatagtgcatccagcagggccactggcatcccagacaggttcagtggcagtgggtctgggacagacttcactctcaccatcagcagactggagcctgaagattttgcagtgtattactgtcagcagtatggtagctcacctcccatgtacacttttggccaagggaccaag

SEQ ID NO: 61 - DNA, 291 bp, Homo sapiens
tctccaggcaccctgtctttgtctccaggggaaagagccaccctctcctgcagggccagtcagagtgttagcagcacctacttagcctggtaccagcagaaacctggccaggctcctaggctcctcatctatgatgcatccagcagggccactggcatcccagacaggttcagtggcagtgggtctgggacagacttcactctcaccatcagcagactggagcctgaagattttgcagtgtattactgtcagcagtctggtaacttaatcactttcggccaagggaccaag

SEQ ID NO: 62 - DNA, 300 bp, Homo sapiens
tctccaggcaccctgtctttgtctccaggggaaagagccaccctctcctgcagggccagtcagagtgttagcggcagctacttagcctggtaccagcagaaacctggccaggctcctaggctcctcatctatgatgcatccagcagggccactggcatcccagacaggttcagtggcagtgggtctgggacagacttcactctcaccatcagcagactggagcctgaagattttgcagtgtattactgtcaggtgtatgttagttcacctccggcgtgggcgttcggccaagggaccaag

SEQ ID NO: 63 - DNA, 20 bp, Artificial Sequence
  Description of Artificial Sequence Synthetic primer
tttttttttttttttttttt

SEQ ID NO: 64 - DNA, 27 bp, Homo sapiens
agtcagagtgttcgcggcaggtactta

SEQ ID NO: 65 - DNA, 27 bp, Homo sapiens
agtgagagtgttagcgacagctactta

SEQ ID NO: 66 - DNA, 27 bp, Homo sapiens
agtgagagtgttagcaacacctactta

SEQ ID NO: 67 - DNA, 27 bp, Homo sapiens
agtcagagtgtcagcgacagctactta

SEQ ID NO: 68 - DNA, 27 bp, Homo sapiens
agtcagagtcttactagcacctactta

SEQ ID NO: 69 - DNA, 27 bp, Homo sapiens
agtcagagtcttactagcagctactta

SEQ ID NO: 70 - DNA, 27 bp, Homo sapiens
agtgagagtgttagcaacacctactta

SEQ ID NO: 71 - DNA, 27 bp, Homo sapiens
agtcagactattatcagcaccaactta

SEQ ID NO: 72 - DNA, 27 bp, Homo sapiens
agtcagactattagcagcacctactta

SEQ ID NO: 73 - DNA, 27 bp, Homo sapiens
agtcagagtcttagcagcagcaactta

SEQ ID NO: 74 - DNA, 27 bp, Homo sapiens
agtcagagtcttaccagcacctactta

SEQ ID NO: 75 - DNA, 27 bp, Homo sapiens
agtcagagtcttaccagcagctactta

SEQ ID NO: 76 - DNA, 27 bp, Homo sapiens
agtgagagtgttagcaacacctactta

SEQ ID NO: 77 - DNA, 30 bp, Homo sapiens
cagcagtatggtagttcacctcggacgttc

SEQ ID NO: 78 - DNA, 30 bp, Homo sapiens
cagctgtttgataggtcaccattcactttc

SEQ ID NO: 79 - DNA, 30 bp, Homo sapiens
cagctgtttgatagctcaccattcactttc